(19) United States
(12) Patent Application Publication
(10) Pub. No.: US 2012/0277434 A1
(43) Pub. Date: Nov. 1, 2012

(54) METHODS FOR SYNTHESIZING QUINOLINONE COMPOUNDS

(76) Inventors: Shaopei Cai, Seattle, WA (US); Joyce Chou, El Cerrito, CA (US); Eric Harwood, Seattle, WA (US); Timothy MacHajewski, Martinez, CA (US); David Ryckman, Bellevue, WA (US); Xiao Shang, Bellevue, WA (US); Shuguang Zhu, Shoreline, WA (US); Augustus O. Okhamafe, Concord, CA (US); Marc S. Tesconi, Monore, NY (US)

(21) Appl. No.: 13/539,872

(22) Filed: Jul. 2, 2012

Related U.S. Application Data
(63) Continuation of application No. 10/982,757, filed on Nov. 5, 2004, now abandoned.
(60) Provisional application No. 60/517,915, filed on Nov. 7, 2003.
(60) Provisional application No. 60/526,425, filed on Dec. 2, 2003.
(60) Provisional application No. 60/526,426, filed on Dec. 2, 2003.
(60) Provisional application No. 60/546,017, filed on Feb. 19, 2004.

(51) Int. Cl. C07D 295/12

(52) U.S. Cl. 544/363

(57) ABSTRACT

A method of synthesizing a substituted or unsubstituted 4-amino-3-benzimidazolyl quinolinone compound includes reacting a first compound having the formula I with a second compound having the formula II in a suitable solvent in the presence of a sodium or potassium salt of a base. The first compound and the second compound have the following structures where the variables have the values described herein:

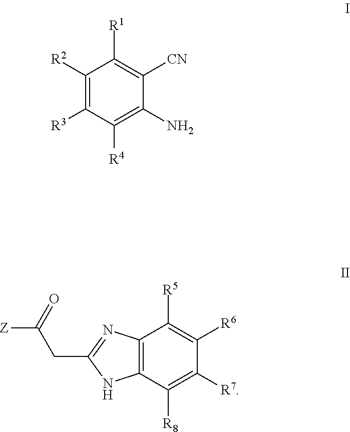

### CROSS REFERENCES TO RELATED APPLICATIONS

This application is a continuation of Ser. No. 10/982,757 filed Nov. 15, 2004, which claims benefit of U.S. provisional Application No. 60/517,915 filed Nov. 7, 2003, U.S. Provisional Application No. 60/526,425 filed Dec. 2, 2003, U.S. Provisional Application No. 60/526,426 filed Dec. 2, 2003, and U.S. Provisional Application No. 60/546,017 filed Feb. 19, 2004, which in their entirety are herein incorporated by reference.

### FIELD OF THE INVENTION

This invention pertains generally to methods of synthesizing quinolinone compounds. More specifically, the invention described herein pertains to improved methods of synthesizing amino quinolinone compounds, and to methods for synthesizing amino quinolinone compounds and compositions that contain low quantities of lithium.

### BACKGROUND OF THE INVENTION

A variety of chemical compounds and compositions have been reported as having activity against one or more vascular endothelial growth factor receptor tyrosine kinase (VEGF-RTK). Examples include quinoline derivatives such as described in WO 98/13350, aminonicotinamide derivatives (see, e.g. WO 01/55114), antisense compounds (see, e.g. WO 01/52904), peptidomimetics (see, e.g. WO 01/52875), quinazoline derivatives (see, e.g. U.S. Pat. No. 6,258,951) monoclonal antibodies (see, e.g. EP 1 086 705 A1), various 5,10,15,20-tetraaryl-porphyrins and 5,10,15-triaryl-corroles (see, e.g. WO 00/27379), heterocyclic alkanesulfonic and alkane carboxylic acid derivatives (see, e.g. DE19841985), oxindolylquinazoline derivatives (see, e.g. WO 99/10349), 1,4-diazaanthracine derivatives (see, e.g. U.S. Pat. No. 5,763,441), and cinnoline derivatives (see, e.g. WO 97/34876), and various indazole compounds (see, e.g. WO 01/02369 and WO 01/53268).

The synthesis of 4-hydroxy quinolone and 4-hydroxy quinoline derivatives is disclosed in a number of references. For example, Ukrainets et al. have disclosed the synthesis of 3-(benzimidazol-2-yl)-4-hydroxy-2-oxo-1,2-dihydroquinoline. Ukrainets, I. et al., Tetrahedron Lett. 42, 7747-7748 (1995); Ukrainets, I. et al., Khimiya Geterotsiklicheskikh Soedinii, 2, 239-241 (1992). Ukrainets has also disclosed the synthesis, anticonvulsive and antithyroid activity of other 4-hydroxy quinolones and thio analogs such as 1H-2-oxo-3-(2-benzimidazolyl)-4-hydroxyquinoline. Ukrainets, I. et al., Khimiya Geterotsiklicheskikh Soedinii, 1, 105-108 (1993); Ukrainets, I. et al., Khimiya Geterotsiklicheskikh Soedinii, 8, 1105-1108 (1993); Ukrainets, I. et al., Chem. Heterocyclic Comp. 33, 600-604, (1997).

The synthesis of various quinoline derivatives is disclosed in WO 97/48694. These compounds are disclosed as capable of binding to nuclear hormone receptors and being useful for stimulating osteoblast proliferation and bone growth. The compounds are also disclosed as being useful in the treatment or prevention of diseases associated with nuclear hormone receptor families.

Various quinoline derivatives in which the benzene ring of the quinoline is substituted with a sulfur group are disclosed in WO 92/18483. These compounds are disclosed as being useful in pharmaceutical formulations and as medicaments.

Quinolone and coumarin derivatives have been disclosed as having use in a variety of applications unrelated to medicine and pharmaceutical formulations. References that describe the preparation of quinolone derivatives for use in photopolymerizable compositions or for luminescent properties include: U.S. Pat. No. 5,801,212 issued to Okamoto et al.; JP 8-29973; JP 7-43896; JP 6-9952; JP 63-258903; EP 797376; and DE 23 63 459.

A plethora of substituted quinolinone compounds including quinolinone benzimidazolyl compounds and 4-amino substituted quinolinone benzimidazolyl compounds such as 4-amino-5-fluoro-3-[5-(4-methylpiperazin-1-yl)-1H-benzimidazol-2-yl]quinolin-2(1H)-one have recently been disclosed in references such as WO 02/22598 and WO 2004/043389. Such compounds are disclosed as inhibiting VEGF-RTKs. Such compounds are also disclosed in published United States patent applications U.S. 2002/0107392 and U.S. 2003/0028018 and U.S. Pat. Nos. 6,605,617, 6,774,237, and 6,762,194. Heterocyclic compounds related to benzimidazolyl quinolinones have recently been disclosed in WO 02/18383, U.S. 2002/0103230, and U.S. Pat. No. 6,756,383. Other such compounds are disclosed along with new uses of such compounds in inhibiting serine/threonine kinases and tyrosine kinases are disclosed in WO 2004/018419, and U.S. 2004/0092535, filed on Aug. 19, 2003, and claiming priority to each of the following provisional applications: U.S. Provisional Application No. 60/405,729 filed on Aug. 23, 2002; U.S. Provisional Application No. 60/426,107 filed on Nov. 13, 2002; U.S. Provisional Application No. 60/426,226 filed on Nov. 13, 2002; U.S. Provisional Application No. 60/426,282 filed on Nov. 13, 2002; U.S. Provisional Application No. 60/428,210 filed on Nov. 21, 2002; U.S. Provisional Application No. 60/460,327 filed on Apr. 3, 2003; U.S. Provisional Application No. filed on Apr. 3, 2003; U.S. Provisional Application No. 60/460,493 filed on Apr. 3, 2003; U.S. Provisional Application No. 60/478,916 filed on Jun. 16, 2003; and U.S. Provisional Application No. 60/484,048 filed on Jul. 1, 2003. Each of the references in this paragraph is hereby incorporated by reference in its entirety and for all purposes as if fully set forth herein.

Although various methods have been disclosed for synthesizing quinolinone compounds, new methods which optimize yields of these compounds are needed because of their important applications in pharmaceutical formulations and applications.

### SUMMARY OF THE INVENTION

The present invention provides methods of synthesizing quinolinone compounds such as amino substituted benzimidazolyl quinolinone compounds. The invention further provides amino substituted benzimidazolyl quinolinone compounds and formulations with reduced quantities of lithium and methods for synthesizing such compounds and formulations that do not require the use of or include lithium salts.

In one aspect, the present invention provides a method of synthesizing a substituted or unsubstituted 4-amino-3-benzimidazolyl quinolinone compound and compositions that include such a compound. The method includes reacting a first compound having the formula I with a second compound having the formula II in a suitable solvent in the presence of a potassium or sodium salt of a base. The reaction of the first compound with the second compound produces the substituted or unsubstituted 4-amino-3-benzimidazolyl quinolinone compound. In some embodiments, the method includes reacting the first compound with the second compound in a suitable solvent in the presence of the potassium salt of the base. Formula I and formula II have the following structures:

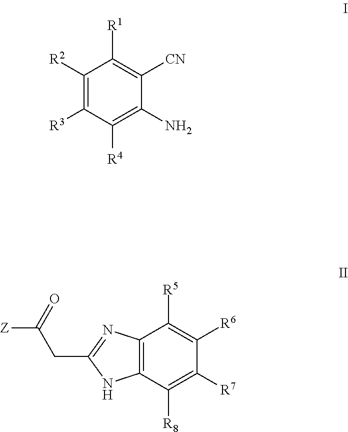

where:

R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;

R⁵, R⁶, R⁷, and R⁸ may be the same or different and are independently selected from H, Cl, Br, F, I, —OR¹³ groups, —NR¹⁴R¹⁵ groups, —SR¹⁶ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

Z is selected from —OR^{9a} groups or —NR^{9b}R^{9c} groups;

R^{9a} is an unsubstituted alkyl group having from 1 to 8 carbon atoms and is absent if Z is a —NR^{9b}R^{9c} group;

R^{9b} and R^{9c} are independently selected from unsubstituted alkyl groups having from 1 to 8 carbon atoms or are both absent if Z is a —OR^{9a} group;

R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹¹ and R¹⁴ may be the same or different and are independently selected from substituted or unsubstituted alkyl groups, substituted or unsubstituted aryl groups, or substituted or unsubstituted heterocyclyl groups;

R¹² and R¹⁵ may be the same or different and are independently selected from substituted or unsubstituted alkyl groups, substituted or unsubstituted aryl groups, or substituted or unsubstituted heterocyclyl groups; and

R¹⁶ is selected from substituted or unsubstituted alkyl groups, substituted or unsubstituted aryl groups, or substituted or unsubstituted heterocyclyl groups.

In some embodiments, the substituted or unsubstituted 4-amino-3-benzimidazolyl quinolinone compound is a compound having the formula III or a tautomer of the compound. Formula III has the following structure.

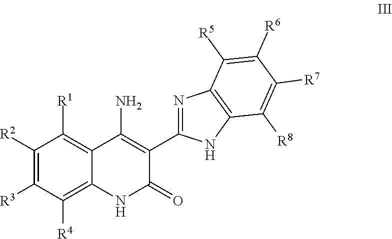

where R¹ through R⁸ and R¹⁶ through R¹⁶ have the values described above.

In some embodiments, the method further includes reacting the substituted or unsubstituted 4-amino-3-benzimidazolyl quinolinone compound or a tautomer of the compound with lactic acid, wherein the lactic acid salt of the 4-amino-3-benzimidazolyl quinolinone compound or the tautomer is obtained.

Further objects, features and advantages of the invention will be apparent from the following detailed description.

### DETAILED DESCRIPTION OF THE INVENTION

The present invention provides methods for synthesizing amino substituted quinolinone compounds. Such compounds act as antagonists of receptor tyrosine kinases, and, more particularly, as inhibitors of PDGFRα and PDGFRβ, bFGF and/or VEGF-RTK function. Such compounds also have potent activity with respect to other tyrosine kinases and also with respect to various serine/threonine kinases. The compounds provided herein can be formulated into pharmaceutical formulations that are useful, for example, in treating patients with a need for an inhibitor of VEGF-RTK, especially, for use in compositions and methods for reducing capillary proliferation and in the treatment of cancer. The methods for synthesizing amino substituted quinolinone compounds allows for the synthesis of formulations and compounds that have reduced amounts of lithium.

The following abbreviations and definitions are used throughout this application:

“bFGF” is an abbreviation that stands for basic fibroblast growth factor.

“bFGFR”, also referred to as FGFR1, is an abbreviation that stands for a tyrosine kinase that interacts with the fibroblast growth factor FGF.

“PDGF” is an abbreviation that stands for platelet derived growth factor. PDGF interacts with tyrosine kinases PDGFRα and PDGFRβ.

“RTK” is an abbreviation that stands for receptor tyrosine kinase.

“VEGF” is an abbreviation that stands for vascular endothelial growth factor.

“VEGF-RTK” is an abbreviation that stands for vascular endothelial growth factor receptor tyrosine kinase.

Generally, reference to a certain element such as hydrogen or H is meant to include all isotopes of that element. For example, if an R group is defined to include hydrogen or H, it also includes deuterium and tritium.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cycloalkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms. More preferred such unsubstituted alkyl groups have from 1 to 10 carbon atoms while even more preferred such groups have from 1 to 5 carbon atoms. Most preferred unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. Preferred substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

The phrase “unsubstituted aryl” refers to aryl groups that do not contain heteroatoms. Thus, by way of example, the phrase includes, but is not limited to, groups such as phenyl, biphenyl, anthracenyl, and naphthyl. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. A preferred unsubstituted aryl group is phenyl. In some embodiments, unsubstituted aryl groups have from 6 to 14 carbon atoms. Unsubstituted aryl groups may be bonded to one or more carbon atom(s), oxygen atom(s), nitrogen atom(s), and/or sulfur atom(s) in the parent compound.

The phrase “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to groups such as tolyl, and hydroxyphenyl among others.

The phrase “unsubstituted alkenyl” refers to straight and branched chain and cyclic groups such as those described with respect to unsubstituted alkyl groups as defined above, except that at least one double bond exists between two carbon atoms. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others. In some embodiments, unsubstituted alkenyl groups have from 2 to 8 carbon atoms.

The phrase “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

The phrase “unsubstituted alkynyl” refers to straight and branched chain groups such as those described with respect to unsubstituted alkyl groups as defined above, except that at least one triple bond exists between two carbon atoms. Examples include, but are not limited to —C≡C(H), —C≡C(CH₃), —C≡C(CH₂CH₃), —C(H₂)C≡C(H), —C(H)₂C≡C(CH₃), and —C(H)₂C≡C(CH₂CH₃) among others. In some embodiments, unsubstituted alkynyl groups have from 2 to 8 carbon atoms.

The phrase “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The phrase “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridinyl, dihydropyridinyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiophene, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

The phrase “substituted heterocyclyl” refers to an unsubstituted heterocyclyl group as defined above in which one or more of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, N-alkyl piperazinyl groups such as 1-methyl piperazinyl, piperazine-N-oxide, N-alkyl piperazine N-oxides, 2-phenoxy-thiophene, and 2-chloropyridinyl among others. In addition, substituted heterocyclyl groups also include heterocyclyl groups in which the bond to the non-hydrogen atom is a bond to a carbon atom that is part of a substituted and unsubstituted aryl, substituted and unsubstituted aralkyl, or unsubstituted heterocyclyl group. Examples include but are not limited to 1-benzylpiperidinyl, 3-phenythiomorpholinyl, 3-(pyrrolidin-1-yl)-pyrrolidinyl, and 4-(piperidin-1-yl)-piperidinyl. Groups such as N-alkyl substituted piperazine groups such as N-methyl piperazine, substituted morpholine groups, and piperazine N-oxide groups such as piperazine N-oxide and N-alkyl piperazine N-oxides are examples of some substituted heterocyclyl groups. Groups such as substituted piperazine groups such as N-alkyl substituted piperazine groups such as N-methyl piperazine and the like, substituted morpholine groups, piperazine N-oxide groups, and N-alkyl piperazine N-oxide groups are examples of some substituted heterocyclyl groups that are especially suited as R⁶ or R⁷ groups.

The phrase “unsubstituted heterocyclylalkyl” refers to unsubstituted alkyl groups as defined above in which a hydrogen or carbon bond of the unsubstituted alkyl group is replaced with a bond to a heterocyclyl group as defined above. For example, methyl (—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group. In addition, a substituted heterocyclylalkyl group also includes groups in which a carbon bond or a hydrogen bond of the alkyl part of the group is replaced by a bond to a substituted and unsubstituted aryl or substituted and unsubstituted aralkyl group. Examples include but are not limited to phenyl-(piperidin-1-yl)-methyl and phenyl-(morpholin-4-yl)-methyl.

The phrase “unsubstituted alkoxy” refers to a hydroxyl group (—OH) in which the bond to the hydrogen atom is replaced by a bond to a carbon atom of an otherwise unsubstituted alkyl group as defined above.

The phrase “substituted alkoxy” refers to a hydroxyl group (—OH) in which the bond to the hydrogen atom is replaced by a bond to a carbon atom of an otherwise substituted alkyl group as defined above.

The phrase “unsubstituted heterocyclyloxy” refers to a hydroxyl group (—OH) in which the bond to the hydrogen atom is replaced by a bond to a ring atom of an otherwise unsubstituted heterocyclyl group as defined above.

The phrase “substituted heterocyclyloxy” refers to a hydroxyl group (—OH) in which the bond to the hydrogen atom is replaced by a bond to a ring atom of an otherwise substituted heterocyclyl group as defined above.

The phrase “unsubstituted aryloxyalkyl” refers to an unsubstituted alkyl group as defined above in which a carbon bond or hydrogen bond is replaced by a bond to an oxygen atom which is bonded to an unsubstituted aryl group as defined above.

The phrase “substituted aryloxyalkyl” refers to an unsubstituted aryloxyalkyl group as defined above in which a bond to a carbon or hydrogen group of the alkyl group of the aryloxyalkyl group is bonded to a non-carbon and non-hydrogen atom as described above with respect to substituted alkyl groups or in which the aryl group of the aryloxyalkyl group is a substituted aryl group as defined above.

The phrase “unsubstituted heterocyclyloxyalkyl” refers to an unsubstituted alkyl group as defined above in which a carbon bond or hydrogen bond is replaced by a bond to an oxygen atom which is bonded to an unsubstituted heterocyclyl group as defined above.

The phrase “substituted heterocyclyloxyalkyl” refers to an unsubstituted heterocyclyloxyalkyl group as defined above in which a bond to a carbon or hydrogen group of the alkyl group of the heterocyclyloxyalkyl group is bonded to a non-carbon and non-hydrogen atom as described above with respect to substituted alkyl groups or in which the heterocyclyl group of the heterocyclyloxyalkyl group is a substituted heterocyclyl group as defined above.

The phrase “unsubstituted heterocyclylalkoxy” refers to an unsubstituted alkyl group as defined above in which a carbon bond or hydrogen bond is replaced by a bond to an oxygen atom which is bonded to the parent compound, and in which another carbon or hydrogen bond of the unsubstituted alkyl group is bonded to an unsubstituted heterocyclyl group as defined above.

The phrase “substituted heterocyclylalkoxy” refers to an unsubstituted heterocyclylalkoxy group as defined above in which a bond to a carbon or hydrogen group of the alkyl group of the heterocyclylalkoxy group is bonded to a non-carbon and non-hydrogen atom as described above with respect to substituted alkyl groups or in which the heterocyclyl group of the heterocyclylalkoxy group is a substituted heterocyclyl group as defined above. Further, a substituted heterocyclylalkoxy group also includes groups in which a carbon bond or a hydrogen bond to the alkyl moiety of the group may be substituted with one or more additional substituted and unsubstituted heterocycles. Examples include but are not limited to pyrid-2-ylmorpholin-4-ylmethyl and 2-pyrid-3-yl-2-morpholin-4-ylethyl.

The phrase “unsubstituted alkoxyalkyl” refers to an unsubstituted alkyl group as defined above in which a carbon bond or hydrogen bond is replaced by a bond to an oxygen atom which is bonded to an unsubstituted alkyl group as defined above.

The phrase “substituted alkoxyalkyl” refers to an unsubstituted alkoxyalkyl group as defined above in which a bond to a carbon or hydrogen group of the alkyl group and/or the alkoxy group of the alkoxyalkyl group is bonded to a non-carbon and non-hydrogen atom as described above with respect to substituted alkyl groups.

The term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999) which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals; and others.

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Generally, the invention provides methods for synthesizing benzimidazolyl quinolinone compounds such as amino substituted benzimidazolyl quinolinone compounds. The invention further provides amino substituted benzimidazolyl quinolinone compounds and formulations that have reduced amounts of lithium and methods of synthesizing such compounds and compositions.

In one aspect, the present invention provides a method for synthesizing a substituted or unsubstituted 4-amino-3-benzimidazolyl quinolinone compound and compositions that include such a compound. The method includes reacting a first compound having the formula I with a second compound having the formula II in a suitable solvent in the presence of a sodium or potassium salt of a base. In some embodiments, the method includes reacting the first compound with the second compound in the suitable solvent in the presence of the potassium salt of the base. The reaction of the first compound with the second compound produces the substituted or unsubstituted 4-amino-3-benzimidazolyl quinolinone compound. Formula I and formula II have the following structures:

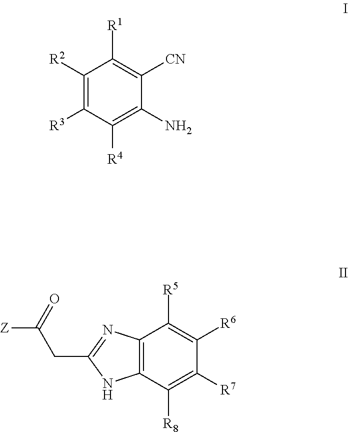

where:

R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;

R⁵, R⁶, R⁷, and R⁸ may be the same or different and are independently selected from H, Cl, Br, F, I, —OR¹³ groups, —NR¹⁴R¹⁵ groups, —SR¹⁶ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

Z is selected from —OR^{9a} groups or —NR^{9b}R^{9c} groups;

R^{9a} is an unsubstituted alkyl group having from 1 to 8 carbon atoms and is absent if Z is a —NR^{9b}R^{9c} group;

R^{9b} and R^{9c} are independently selected from unsubstituted alkyl groups having from 1 to 8 carbon atoms or are both absent if Z is a —OR^{9a} group;

R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹¹ and R¹⁴ may be the same or different and are independently selected from substituted or unsubstituted alkyl groups, substituted or unsubstituted aryl groups, or substituted or unsubstituted heterocyclyl groups;

R¹² and R¹⁵ may be the same or different and are independently selected from substituted or unsubstituted alkyl groups, substituted or unsubstituted aryl groups, or substituted or unsubstituted heterocyclyl groups; and

R¹⁶ is selected from substituted or unsubstituted alkyl groups, substituted or unsubstituted aryl groups, or substituted or unsubstituted heterocyclyl groups.

In some embodiments, the substituted or unsubstituted 4-amino-3-benzimidazolyl quinolinone compound is a compound having the formula III, is a tautomer of the compound having the formula III, is a salt of the compound having the formula III, or is a salt of the tautomer of the compound having the formula III. Formula III has the following structure:

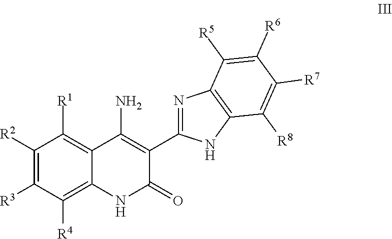

where R¹ through R⁸ and R¹⁰ through R¹⁶ have the values described above.

In some embodiments of the method, R¹ is selected from H, Cl, Br, F, or I. In some such embodiments, R¹ is F. In some specific embodiments, R¹ is F and each of R², R³ and R⁴ is H such that the first compound is a compound having the formula IA which has the following structure

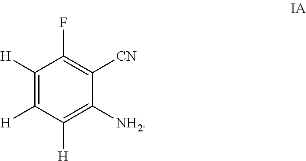

In other embodiments, at least one of R⁶ or R⁷ is a substituted or unsubstituted heterocyclyl group. In some such embodiments, one of R⁶ or R⁷ is a heterocyclyl group and the other of R⁶ or R⁷ is a H. In some embodiments, one of R⁶ or R⁷ is a heterocyclyl group selected from a substituted or unsubstituted piperidinyl group, piperazinyl group, or morpholinyl group. In some such embodiments one of R⁶ or R⁷ is an N-alkyl piperazinyl group such as an N-methyl piperazinyl group or the like and, in some such embodiments, the other of R⁶ or R⁷ is a H. In other such embodiments, Z is an —OR^{9a} group. Therefore, in some embodiments, the second compound is a compound having the formula IIA or IIB and has one of the following structures where R⁵, R⁸, and R^{9a} have the values described above for compounds having the formula II.

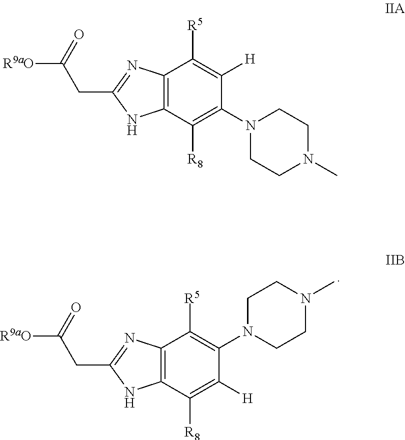

In some further embodiments, the second compound is a compound having the formula IIA or IIB and both R⁵ and R⁸ are H such that the second compound is a compound having the formula IIC or IID and has one of the following structures.

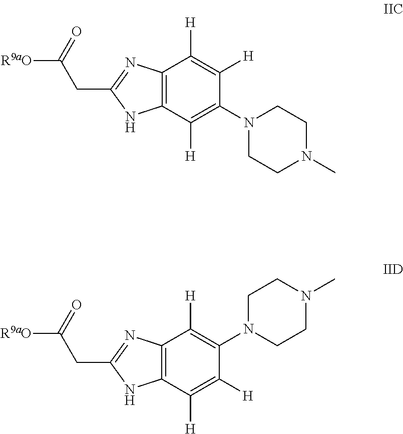

In some embodiments of the method, R^{9a}, R^{9b}, and R^{9c} are straight chain alkyl groups selected from methyl, ethyl, propyl, butyl, or pentyl groups or are branched chain alkyl groups selected from i-propyl, s-butyl, or t-butyl groups. In some embodiments, R^{9a}, R^{9b}, or R^{9c} are methyl, ethyl, or propyl groups and in yet other embodiments, R^{9a}, R^{9b}, or R^{9c} are ethyl groups.

In some embodiments of the method, the method includes reacting the first compound with the second compound in a solvent such as a dialkyl ether such as, but not limited to, diethyl ether or the like; a cyclic ether such as, but not limited to, dioxane, tetrahydrofuran or the like; an aromatic solvent such as toluene, o-xylene, m-xylene, p-xylene, mixtures thereof, or the like; or combinations of these solvents. Other suitable solvents include polar aprotic solvents such as DMF (N,N-Dimethylformamide) and the like. In some such embodiments, the solvent is tetrahydrofuran. In other embodiments, the solvent is toluene. In some embodiments, the concentration of the first compound is greater than or about 0.10 M or is greater than or about 0.15 M based on the amount of the solvent when the first compound and the second compound are reacted. In some such embodiments, the concentration of the first compound ranges from 0.10 M to 0.30 M based on the amount of solvent when the first compound and the second compound are reacted. In some such embodiments, the concentration of the first compound ranges from 0.15 M to 0.25 M based on the amount of solvent when the first compound and the second compound are reacted. In some such embodiments, the concentration of the first compound ranges from 0.17 M to 0.22 M based on the amount of solvent when the first compound and the second compound are reacted. In some such embodiments, the concentration of the first compound is about 0.19 M based on the amount of solvent when the first compound and the second compound are reacted. In some such embodiments, the concentration of the first compound and/or the second compound ranges from 0.15 M to 0.50 M based on the amount of solvent when the first compound and the second compound are reacted. In some such embodiments, the concentration of the first compound and/or the second compound ranges from 0.20 M to 0.45 M based on the amount of solvent when the first compound and the second compound are reacted. In some such embodiments, the concentration of the first compound and/or the second compound ranges from 0.25 M to 0.45 M based on the amount of solvent when the first compound and the second compound are reacted. In some embodiments, the concentration of the second compound is greater than 0.10 M based on the amount of the solvent when the first compound and the second compound are reacted. In other such embodiments, the concentration of the second compound is greater than about 0.15 M, whereas in other embodiments, the concentration of the second compound is greater than about 0.20 M based on the amount of solvent when the first compound and the second compound are reacted. In some embodiments, the concentration of the second compound ranges from 0.15 M to 0.30 M based on the amount of solvent when the first compound and the second compound are reacted. In some embodiments, the concentration of the second compound ranges from 0.18 M to 0.26 M based on the amount of solvent when the first compound and the second compound are reacted. In some embodiments, the concentration of the second compound ranges from 0.20 M to 0.24 M based on the amount of solvent when the first compound and the second compound are reacted. In some embodiments, the concentration of the second compound is about 0.22 M based on the amount of solvent when the first compound and the second compound are reacted. In some embodiments, the solvent is dried prior to use in the reaction. In some such embodiments, the solvent of the reaction comprises, less than 0.5 percent water, less than 0.25 percent water, less than 0.1 percent water, or is less than 0.05 percent water by weight. In still other such embodiments, the solvent comprises less than 0.01 percent water, or is less than 0.005 percent water based on the weight. In some embodiments, the solvent is dried prior to use in the reaction. In some embodiments, a mixture of the solvent and the second compound is dried prior to addition of the potassium or sodium salt of the base. In some such embodiments, the mixture of the solvent and the second compound comprises, less than 0.5 percent water, less than 0.25 percent water, less than 0.2 percent water, less than 0.1 percent water, or less than 0.05 percent water which may be determined by Karl Fischer analysis.

In some embodiments of the method, the method includes reacting the first compound with the second compound in the suitable solvent using the sodium or potassium salt of a base that may be used to generate an enolate anion, which, in some embodiments, may be a sterically-hindered base. As used herein, the term “base” refers to a chemical compound that deprotonates another compound when reacted with it. In some such embodiments, the sodium or potassium salt of the base that may be used to generate an enolate anion is a base such as, for example, NaH, KH, Na₂CO₃, K₂CO₃, sodium and potassium alkoxides such as, but not limited to, sodium and potassium t-butoxide, propoxide, i-propoxide, ethoxide, methoxide, and the like, sodium amide (NaNH₂), potassium amide (KNH₂), and the like. In some embodiments, the base is sodium or potassium t-butoxide, and in some such embodiments, the base is potassium t-butoxide in a solvent such as THF. In some of these embodiments the base is potassium t-butoxide (20% in THF). In some embodiments, the sterically hindered base is an amide anion and in some such embodiments, the amide nitrogen is bonded to two trialkylsilyl groups. In some such embodiments, the sodium or potassium salt of the base is selected from a sodium or potassium bis(trialkylsilyl)amide. In some such embodiments, the sodium or potassium bis(trialkylsilyl)amide is sodium bis(trimethylsilyl)amide (NaHMDS) or potassium bis(trimethylsilyl)amide (KHMDS). In some embodiments, the method further includes adding the sodium or potassium salt of the base to a mixture of the first compound and the second compound in the suitable solvent. In some embodiments, the sodium or potassium salt of the base is present in an amount of from 2 to 4 equivalents, and in some such embodiments in an amount of from 2.5 to 3 equivalents, with respect to the first compound. In still other embodiments, the sodium or potassium salt of the base is present in an amount of 2 to 4 equivalents, and in some such embodiments in an amount of from 2.5 to 3 equivalents, with respect to the second compound. In some embodiments, the second compound is present in an amount of from 1 to 2 equivalents with respect to the first compound. In some such embodiments, the second compound is present in an amount of from 1 to 1.5 equivalents with respect to the first compound.

In some embodiments of the method for synthesizing a substituted or unsubstituted 4-amino-3-benzimidazole quinolinone compound and compositions that include such compounds, the method includes adding the potassium salt of the base to a mixture comprising the first compound, the second compound, and the suitable solvent at a temperature of from 20° C. to 50° C. In some such embodiments, the potassium salt of the base is added to the mixture and the temperature of the mixture is from 25° C. to 45° C., from 35° C. to 45° C., or from 38° C. to 42° C. when the potassium salt of the base is first added to the mixture. In some embodiments, the internal temperature is 40° C. or about 40° C. when the potassium salt of the base is first added to the mixture. The internal reaction temperature generally increases, for example up to 62° C. or 65° C. upon addition of the potassium salt of the base to the reaction mixture. However, in some embodiments, the internal temperature is maintained at 30° C. to 52° C., 36° C. to 52° C., or in some embodiments from 38° C. to 50° C. during addition of the potassium salt of the base. In some such embodiments, the potassium salt of the base is added to the mixture over a period of from 2 to 20 minutes. In some such embodiments, the potassium salt of the base is added to the mixture over a period of from 3 to 10 minutes and in some such embodiments, the potassium salt of the base is added to the mixture over a period of from 5 to 10 minutes or in some embodiments over a period of 5 minutes.

In some embodiments of the method for synthesizing a substituted or unsubstituted 4-amino-3-benzimidazole quinolinone compound and compositions that include such compounds, the method includes adding the sodium or potassium salt of the base to a mixture comprising the first compound, the second compound, and the suitable solvent at a temperature of from 15° C. to 50° C. In some such embodiments, the potassium salt of the base is added to the mixture and the temperature of the mixture is from 15° C. to 25° C., from 15° C. to 20° C., or from 17° C. to 20° C. when the potassium salt of the base is first added to the mixture. In some embodiments, the internal temperature is 17° C. to 20° C. when the potassium salt of the base is first added to the mixture. In some embodiments, the internal temperature is maintained at a temperature of less than or about 25° C. during addition of the base. In some such embodiments, the internal temperature of the reaction is raised to 30° C. and the reaction is monitored for completion using HPLC.

In some embodiments, the method further includes (a) adding an aromatic solvent such as toluene to a reaction flask to provide a reaction mixture comprising the first compound and the second compound; (b) distilling at least a portion of the aromatic solvent from the reaction flask, and (c) repeating (a) and (b) until the water content is less than 0.1 percent, 0.05, 0.04 percent, or 0.03 percent which may be determined using Karl Fischer analysis. In some embodiments, the distillation may be conducted under reduced pressure. In some embodiments, the second compound is dried by (a) mixing the second compound with a suitable organic solvent such as THF, toluene, ethanol, or the like to form a solution, (b) concentrating the second compound by removing at least a portion of the solvent, and (c) optionally repeating steps (a) and (b) one or more additional times. In some such embodiments, (a) and (b) are repeated until the water content of the solution is less than 0.5%, less than 0.4%, less than 0.3%, less than 0.25%, less than 0.20%, less than 0.10%, less than 0.05%, or less than 0.03% which may be determined by Karl Fischer analysis. In some embodiments, steps (a) and (b) are accomplished at least four times. In some embodiments, the second compound may be dried in a reaction vessel and when the desired quantity of drying is achieved, such as a water level of less than 0.25% or less than 0.20%, the first compound and the potassium or sodium salt of the base are added to the reaction vessel. In such embodiments, solvents such as those suitable for use in the reaction of the first compound with the second compound may be used to dry the second compound. Such solvents include ethereal solvents such as diethyl ether, dioxane, THF, and the like and aromatic solvents such as toluene.

In some embodiments of the method for synthesizing a substituted or unsubstituted 4-amino-3-benzimidazolyl quinolinone compound and compositions that include such a compound, the method includes drying the second compound to a water level of less than 5.5 percent by weight prior to reacting it with the first compound or adding it to a reaction vessel containing the first compound or the suitable solvent. In some such embodiments, the second compound is dried to a water level of less than 5 percent by weight, less than 4 percent by weight, less than 3 percent by weight, less than 2.5 percent by weight, less than 2 percent by weight, less than 1 percent by weight, or less than 0.5 percent by weight. In some such embodiments, the second compound may be dried by mixing the hydrated second compound with an organic solvent such as THF, toluene, or ethanol to form a solution, concentrating the solution by solvent removal, and drying the resulting composition under vacuum with heating. In some such embodiments, the second compound is dried by: (a) mixing the hydrated second compound with an organic solvent such as THF, toluene, or ethanol to form a solution, (b) concentrating the second compound by removing at least a portion of the solvent, (c) optionally repeating steps (a) and (b) one or more additional times, and then (d) drying the resulting composition under vacuum with heating.

In some embodiments of the method for synthesizing a substituted or unsubstituted 4-amino-3-benzimidazolyl quinolinone compound and compositions that include such a compound, the method includes reacting the first compound with the second compound in the presence of the sodium or potassium salt of the base for a period of time ranging from 30 minutes to 360 minutes, from 120 minutes to 300 minutes, from 180 to 300 minutes, from 180 minutes to 270 minutes, from 210 minutes to 270 minutes, or from 210 minutes to 240 minutes at a temperature suitable to provide the desired benzimidazolyl quinolinone compound. In some embodiments, the reaction product mixture of the substituted or unsubstituted 4-amino-3-benzimidazolyl quinolinone compound produced by the reaction of the first compound with the second compound is quenched by pouring the reaction product mixture into water. In other embodiments, water is added to the reaction mixture which, in some embodiments, is cooled to a temperature of from 20° C. to 35° C. or from 20° C. to 35° C. prior to adding the water. In some embodiments, solvent may be removed under vacuum after water is added and then additional water is added prior to collection of the solid by filtration. The quenched reaction product mixture is typically filtered and washed with water providing the 4-amino-3-benzimidazolyl quinolinone compound, and in some embodiments, the quenched reaction product may be cooled to a temperature of 5° C. to 10° C. prior to filtration although this is not necessary. In some embodiments, the collected product may be dried under vacuum to produce a yield of greater than 30 percent, greater than 40 percent, greater than 50 percent, greater than 60 percent, greater than 70 percent, or greater than 80 percent of the 4-amino-3-benzimidazolyl quinolinone compound. Some embodiments of the method may further include: (a) mixing the collected product with ethanol; (b) heating the ethanolic mixture for a period of from 10 minutes to 180 minutes, of from 30 minutes to 120 minutes, or of about 60 minutes at a temperature of from 40° C. to 78° C., of from 45° C. to 78° C., of from 60° C. to 78° C., or a reflux temperature; (c) cooling the mixture to a temperature of less than 40° C., less than 35° C., less than 30° C., or less than 20° C.; (d) and filtering the cooled mixture. However, it is not necessary that the mixture be cooled prior to filtration. In some such embodiments the filtered product may be washed with a solvent such as ethanol or water. The resulting product may be dried under vacuum with heating such as in a vacuum oven, a drying pistol, a rotary evaporator, or the like.

In some embodiments of the method for synthesizing a substituted or unsubstituted 4-amino-3-benzimidazolyl quinolinone compound and compositions that include such a compound, the method includes reacting a compound having the formula IV with a compound having the formula V to provide the second compound where the variables R⁵, R⁶, R⁷, R⁸, and R^{9a} have the values set forth above with respect to the second compound having the formula II and X is a halogen atom such as F, Cl, Br, or I, or is the conjugate base of an acid.

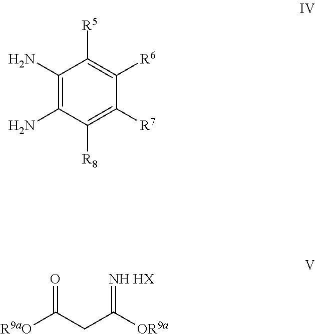

In some such embodiments, the compound having the formula IV has the formula IVA.

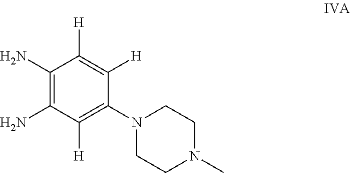

In some such embodiments, the compound having the formula V has the formula VA.

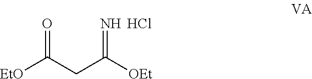

In some embodiments, the compound having the formula IV is reacted with the compound having the formula V in a solvent such as an alcohol such as, but not limited to, ethanol at an internal temperature of from 30° C. to 70° C., of from 35° C. to 60° C., or of from 40° C. to 50° C. for a period of time of from 45 minutes to 240 minutes, of from 60 minutes to 180 minutes, or of from 60 minutes to 120 minutes. In some embodiments, the reaction product from the reaction of the compound having the formula IV with the compound having the formula V is cooled, for example to 25° C. or the like, and is filtered. In other embodiments, the reaction product is still warm when it is filtered through a filter medium such as Celite. In some embodiments, the filter medium may be washed with a solvent such as ethanol, and the filtrate may be concentrated by solvent removal. The concentrated product may then be mixed with an aqueous HCl solution, in some embodiments, a 0.37 percent HCl solution and in other embodiments a 1M HCl solution. A base such as NaOH, for example a 30% NaOH solution, may then be added in one portion or gradually such that a precipitate forms. In some embodiments, the reaction product may be mixed or dissolved with water, in some embodiments deionized water, that is neutral with respect to pH. In such embodiments, the resulting mixture is typically cooled to about 0° C. and then is made basic by addition of a base such as NaOH. In some such embodiments, the pH is brought to about 9.2 by addition of 20% NaOH. In some embodiments, the resulting mixture is stirred for a period of about 1 to 5 hours, for example, for 4 hours or the like, and is then filtered, washed with water and dried in a vacuum oven or the like.

In some embodiments of the method for synthesizing a substituted or unsubstituted 4-amino-3-benzimidazolyl quinolinone compound and compositions that include such a compound, a compound having the formula VIA, VIB, or mixtures thereof is reduced, typically catalytically as described below, with H₂ to produce the compound having the formula IV where the variables R⁵, R⁶, R⁷, and R⁸ have the values set forth above with respect to the second compound having the formula II.

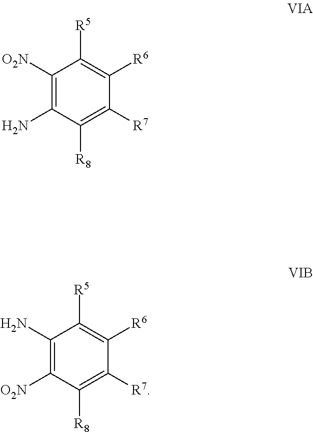

In some such embodiments, the compound having the formula VIA is a compound having the formula VIC or VID and/or the compound having the formula VIB is a compound having the formula VIE or VIF. In some such embodiments, R⁶ or R⁷ is a substituted or unsubstituted heterocyclyl group, that, in some embodiments is selected from substituted or unsubstituted piperidinyl groups, piperazinyl groups, or morpholinyl groups. In some such embodiments, one of R⁶ or R⁷ is an N-alkyl piperazinyl group such as an N-methyl piperazinyl group such that the compounds having the formula VIC, VID, VIE, and VIF have the formula VIG or VIH.

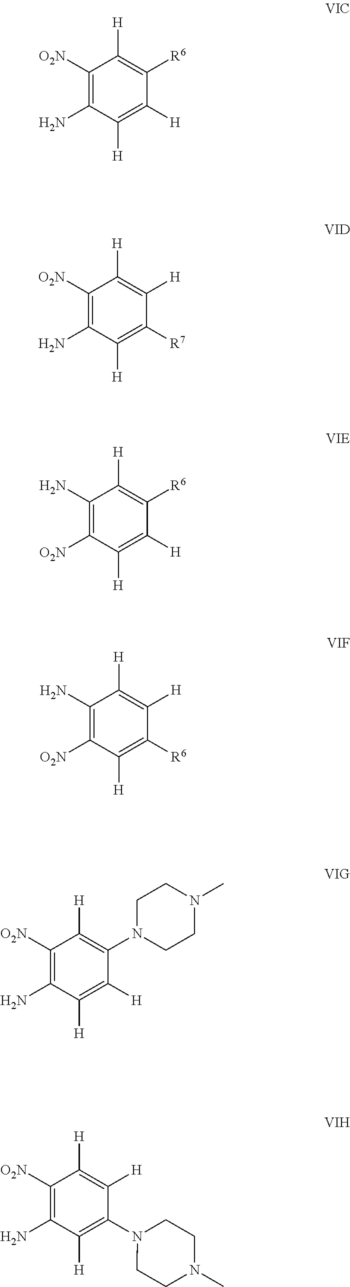

In some embodiments, the compound reduced by H₂ is a compound having the formula VIH. In other embodiments, the compound reduced by H₂ is a compound having the formula VIG. In some embodiments, the compound having the formula VIA, VIB, or mixtures thereof is reduced with H₂ in an alcohol solvent such as ethanol using a transition metal hydrogenation catalyst such as palladium on carbon (Pd/C). In some embodiments, the Pd/C is 5 percent Pd/C and in some embodiments, the Pd/C is 5 percent Pd/C with 50 percent water on a weight by weight basis. In some embodiments, the reaction is conducted at an internal temperature of from 25° C. to 70° C., from 30° C. to 60° C., or in some embodiments from 40° C. to 55° C. or from 45° C. to 55° C. for a period of time of from 1 to 12 hours, of from 3 to 10 hours, of from 4 to 8 hours, or of 6 hours. In some embodiments, the reduced compound having the formula IV is directly reacted with the compound having the formula V in the same reaction vessel without further purification.

In some embodiments of the method for synthesizing a substituted or unsubstituted 4-amino-3-benzimidazolyl quinolinone compound and compositions that include such a compound, a compound having the formula VII is reacted with a compound having the formula HR⁷ or a salt thereof to produce the compound having the formula VIA where the variables R⁵, R⁶, and R⁸ have the values set forth above with respect to the second compound having the formula II and Y is selected from Cl or F.

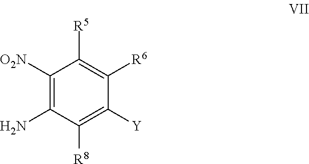

In some such embodiments, the compound having the formula VII is a compound having the formula VIIA or VIIB. In some such embodiments, R⁷ is a substituted or unsubstituted heterocyclyl group, that, in some embodiments is selected from substituted or unsubstituted piperidinyl groups, piperazinyl groups, or morpholinyl groups. In some such embodiments, R⁷ is an N-alkyl piperazinyl group such as an N-methyl piperazinyl group such that HR⁷ has the formula HR⁷(a) shown below.

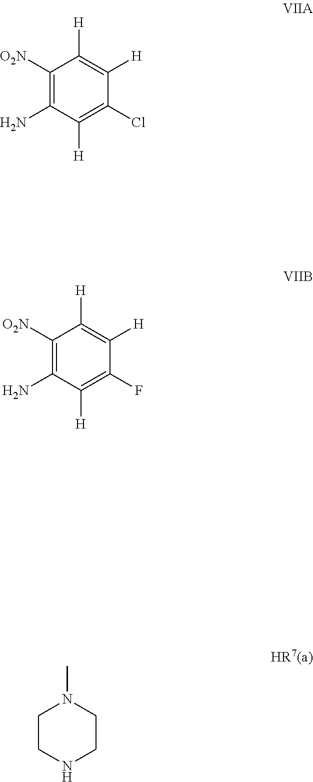

In some embodiments, the compound having the formula VII is reacted with the compound having the formula HR⁷, such as N-methylpiperazine at a temperature of from 70° C. to 120° C. or of 80° C. to 110° C., of from 85° C. to 105° C., or of 100° C. for a period of from 2 hours to 24 hours, of from 4 hours to 12 hours, or of from 6 hours to 10 hours. A variety of suitable solvents such as, but not limited to, ethanol may be employed in the reaction of the compound having the formula HR⁷ with the reaction of the compound having the formula VII. Addition of a solvent such as ethanol to the reaction helps to prevent solidification of the reaction. In some embodiments, any of the reactions of the method are followed by HPLC and are conducted for a period of time until the starting materials are observed to no longer be present in any appreciable amounts.

In some embodiments, the substituted or unsubstituted 4-amino-3-benzimidazolyl quinolinone compound is a compound having the formula IIIA, is a tautomer of the compound having the formula IIIA, is a salt of the compound having the formula IIIA, or is a salt of the tautomer of the compound having the formula IIIA and R⁷ is a substituted or unsubstituted heterocyclyl group

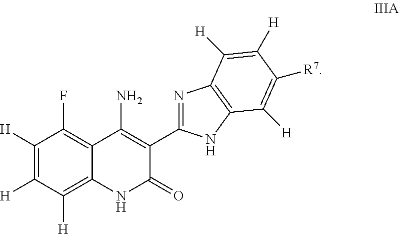

In some such embodiments, R⁷ is a substituted or unsubstituted heterocyclyl group that is selected from a substituted or unsubstituted piperidinyl group, piperazinyl group, or morpholinyl group. In some such embodiments, R⁷ is a substituted or unsubstituted N-alkyl piperazinyl group such as an N-methyl piperazinyl group, an N-ethyl piperazinyl group, or a N-propyl piperazinyl group.

In some embodiments, the substituted or unsubstituted 4-amino-3-benzimidazolyl quinolinone compound is a compound having the formula IIIB, is a tautomer of the compound having the formula IIIB, is a salt of the compound having the formula IIIB, or is a salt of the tautomer of the compound having the formula IIIB

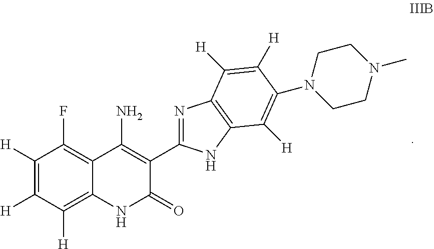

In some embodiments, the method further includes reacting the substituted or unsubstituted 4-amino-3-benzimidazolyl quinolinone compound or a tautomer of the compound with lactic acid, wherein the lactic acid salt of the 4-amino-3-benzimidazolyl quinolinone compound or the tautomer is obtained. In some such embodiments, the compound having the formula IIIB or a tautomer thereof is reacted with lactic acid to produce the lactic acid salt of the compound or tautomer. In some such embodiments, the compound or tautomer is reacted with D,L-lactic acid in water and ethanol and the monolactate salt is produced as a crystalline solid.

The use of a sodium or potassium salt of a base such as, but not limited to, NaHMDS, KHMDS, sodium t-butoxide, or potassium t-butoxide, rather than a lithium salt such as LiHMDS in the reaction of the first compound with the second compound provides a method of producing compositions that include reduced amounts of lithium and in some embodiments may not include any lithium. Furthermore, the use of a base such as potassium t-butoxide results in increased yields of the benzimidazolyl quinolinone compound. Consequently, in some embodiments, the invention provides a composition that includes a benzimidazolyl quinolinone compound having the formula III, a tautomer of the benzimidazolyl quinolinone compound, a salt of the benzimidazolyl quinolinone compound, a salt of the tautomer of the benzimidazolyl compound, or mixtures thereof, wherein the benzimidazolyl quinolinone compound is a compound having the formula III,

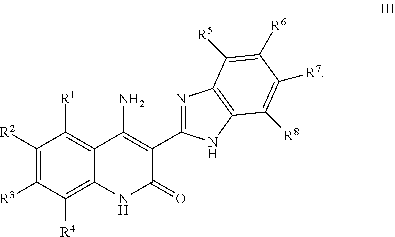

wherein:

R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;

R⁵, R⁶, R⁷, and R⁸ may be the same or different and are independently selected from H, Cl, Br, F, I, —OR¹³ groups, —NR¹⁴R¹⁵ groups, —SR¹⁶ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹¹ and R¹⁴ may be the same or different and are independently selected from substituted or unsubstituted alkyl groups, substituted or unsubstituted aryl groups, or substituted or unsubstituted heterocyclyl groups;

R¹² and R¹⁵ may be the same or different and are independently selected from substituted or unsubstituted alkyl groups, substituted or unsubstituted aryl groups, or substituted or unsubstituted heterocyclyl groups;

R¹⁶ is selected from substituted or unsubstituted alkyl groups, substituted or unsubstituted aryl groups, or substituted or unsubstituted heterocyclyl groups; and further wherein,

the amount of lithium in the composition is less than 1 percent by weight based on the weight of the benzimidazolyl quinolinone compound in the composition.

In some embodiments of the compositions provided herein, the amount of lithium in the composition is less than 0.5 percent, is less than 0.1 percent, is less than 0.05 percent, is less than 0.01 percent, is less than 0.005 percent, or is less than 0.001 by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition. In some such embodiments of the compositions provided herein, lithium is completely absent from the composition. In some embodiments, the composition has less than 1 percent, less than 0.05 percent, or less than 0.01% of the uncyclized intermediate shown in Scheme 1 based on the weight of the benzimidazolyl quinolinone compound.

In some embodiments of the compositions provided herein, the benzimidazolyl quinolinone compound having the formula III is a compound having the formula IIIB

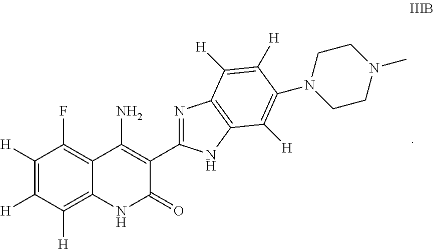

In various groups that include heterocyclyl groups, the heterocyclyl group may be attached in various ways. For example, in an —OCH₂(CH₂)_{q}(heterocyclyl) group, where q is selected from 0, 1, 2, 3, or 4, the heterocyclyl group may be bonded to a methylene carbon of the —OCH₂(CH₂)_{q} group of the —OCH₂(CH₂)_{q}(heterocyclyl) through various ring members. By way of non-limiting example, where q is 1 and the heterocyclyl group is tetrahydrofuran, the group could be represented by the formula —OCH₂CH₂(tetrahydrofuranyl) which corresponds to the following two structures:

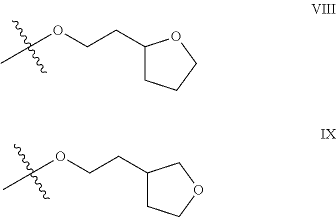

where structure VIII represents the group that can be referred to as the —OCH₂CH₂(2-tetrahydrofuranyl) group and structure IX represents the group that can be referred to as the —OCH₂CH₂(3-tetrahydrofuranyl) group. When the heterocyclyl group is a N-containing heterocycle, such as, but not limited to piperidine, piperazine, morpholine, or pyrrolidine, the heterocycle can be bonded to the methylene carbon through a ring carbon atom or through a nitrogen atom in the ring of the N-containing heterocycle. Both of these are preferred. Where the heterocyclyl group is a piperidine and q is 2 for an —OCH₂(CH₂)_{q}(heterocyclyl) group, the following structures are possible and preferred:

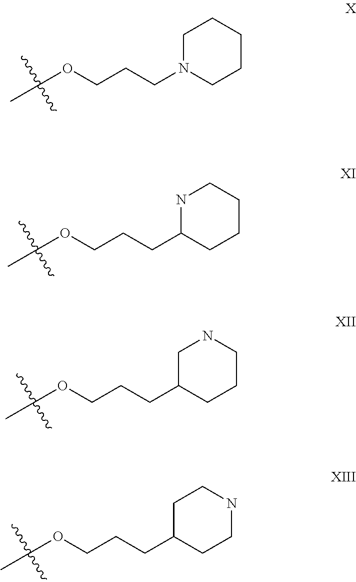

Structure X is an example of a —O(CH₂)₃(N-piperidinyl) or —O(CH₂)₃(1-piperidinyl) group. Structure XI is an example of a —O(CH₂)₃-(2-piperidinyl) group. Structure XII is an example of a —O(CH₂)₃(3-piperidinyl) group. Structure XIII is an example of a —O(CH₂)₃(4-piperidinyl) group. Where the heterocyclyl group is a piperazine and q is 1 for an —OCH₂(CH₂)_{q}(heterocyclyl) group, the following structures are possible and preferred:

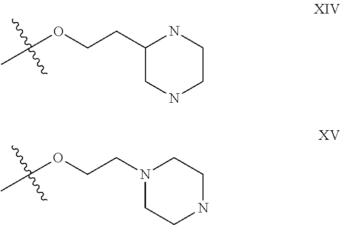

Structure XIV is an example of a —O(CH₂)₂(2-piperazinyl) group, and structure XV is an example of a —O(CH₂)₂(1-piperazinyl) or —O(CH₂)₂(N-piperazinyl) group. Where the heterocyclyl group is a morpholine and q is 1 for an —OCH₂(CH₂)_{q}(heterocyclyl) group, the following structures are possible and preferred:

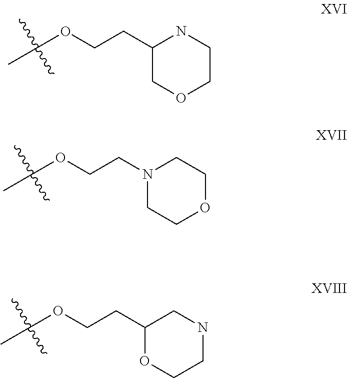

Structure XVI is an example of a —O(CH₂)₂(3-morpholinyl) group, structure XVII is an example of a —O(CH₂)₂(4-morpholinyl) or —O(CH₂)₂(N-morpholinyl) group, and structure XVIII is an example of a —O(CH₂)₂(2-morpholinyl) group. It will be observed that where the group is a pyrrolidine, and q is 1, the structures available include —O(CH₂)₂(1-pyrrolidinyl) or —O(CH₂)₂(N-pyrrolidinyl), —O(CH₂)₂(2-pyrrolidinyl), and —O(CH₂)₂(3-pyrrolidinyl).

Scheme 1 depicts one exemplary synthetic route for the synthesis of a compound of a benzimidazolyl quinolinone compound and should not be interpreted to limit the invention in any manner. As shown below, the reaction of a first compound with a second compound is believed to proceed via an uncyclized intermediate. However, this will be understood to not limit the invention in any manner. The potassium salt of the resulting compound having the formula III produced on cyclization of the intermediate has been found to have reduced solubility resulting in precipitation of the product from the reaction. This was surprising and unexpected given that precipitation was not observed when a lithium salt such as LiHMDS was used rather than a potassium salt such as KHMDS. The use of the potassium salt rather than a lithium salt provides a greatly enhanced yield of compounds having the formula III such as compounds having the formula IIIB as shown in Scheme 1 especially when a base such as a potassium alkoxide such as potassium t-butoxide is employed. The reaction of the first compound with the second compound was also found to provide significantly higher yields of compounds having the formula III when the reaction was conducted with solvents and reactants with low water contents. For example, the yield was found to improve significantly when the second compound was dried as described herein such as by azeotropic evaporation from absolute ethanol or in the reaction vessel by repeated addition of THF followed by distillation. The yield of the compound having the formula VI, such as a compound having the formula VIH, produced by the reaction of an N-alkyl piperazine such as N-methyl piperazine with the compound having the formula VII, was increased when the temperature was lowered and the amount of the compound having the formula HR⁷ was increased with respect to the compound having the formula VI. The temperatures of the reaction were lowered and the reaction was diluted with ethanol during scale up. For example, good yields were obtained when the reaction was conducted at a temperature of 90° C. to 100° C., and the compound having the formula HR⁷, such as N-methyl piperazine, was present in an amount of greater than 2.5 equivalents with respect to the amount of the compound having the formula VI, such as 5-chloro-2-nitroaniline. In some such embodiments, the compound having the formula HR⁷ is present in an amount of greater than 2.8, greater than 2.9, greater than 3.0, or from 2.5 to 5 equivalents with respect to the amount of the compound having the formula VI.

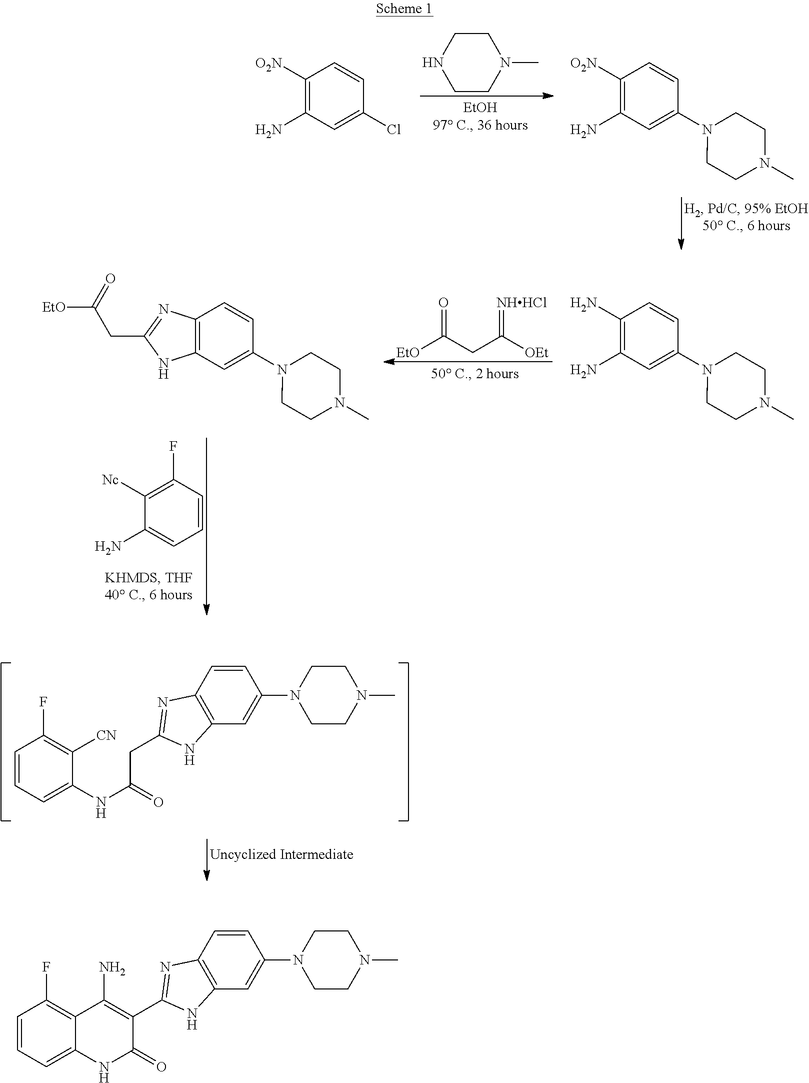

Scheme 2 depicts a method for synthesizing a compound having the formula VA and shows the general application of the method of the invention. Those skilled in the art will understand that the selection of a substituted or unsubstituted diaminobenzene and a substituted or unsubstituted anthranilonitrile allows for the synthesis of a wide variety of compounds having the formula III. Those skilled in the art will also recognize that certain groups may need protection using standard protecting groups for the final cyclization reaction. The extremely versatile synthetic route allows a plethora of compounds having the formula III to be readily prepared by a highly convergent and efficient synthetic route.

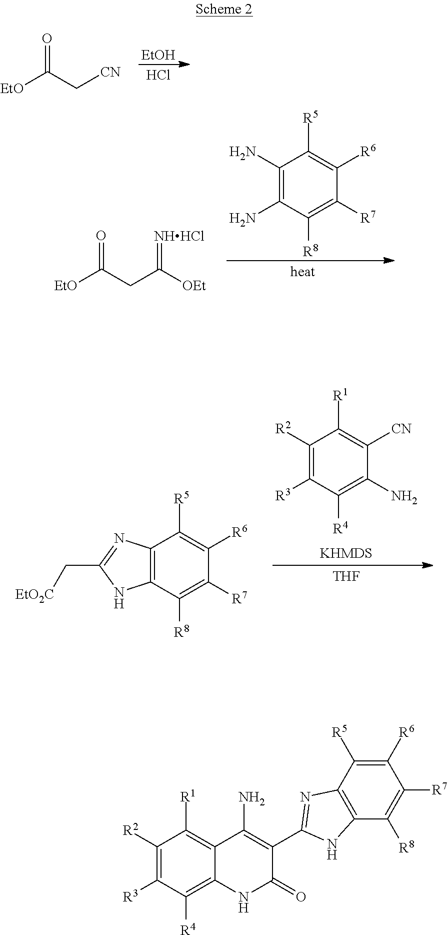

The present invention, thus generally described, will be understood more readily by reference to the following examples, which are provided by way of illustration and are not intended to be limiting of the present invention. The following documents including the examples in the documents are hereby incorporated by reference for all purposes as if fully set forth herein in their entirety: U.S. Pat. No. 6,605,617; U.S. Patent Publication No. 2004/0092535, filed on Aug. 19, 2003; U.S. Provisional Application No. 60/405,729 filed on Aug. 23, 2002; U.S. Provisional Application No. 60/426,107 filed on Nov. 13, 2002; U.S. Provisional Application. No. 60/426,226 filed on Nov. 13, 2002; U.S. Provisional Application No. 60/426,282 filed on Nov. 13, 2002; U.S. Provisional Application No. 60/428,210 filed on Nov. 21, 2002; U.S. Provisional Application No. 60/460,327 filed on Apr. 3, 2003; U.S. Provisional Application No. filed on Apr. 3, 2003; U.S. Provisional Application No. 60/460,493 filed on Apr. 3, 2003; U.S. Provisional Application No. 60/478,916 filed on Jun. 16, 2003; U.S. Provisional Application No. 60/484,048 filed on Jul. 1, 2003, and U.S. Provisional Application No. 60/517,915 filed on Nov. 7, 2003.

### EXAMPLES

The following abbreviations are used in the Examples:

### EtOH: Ethanol

### H₂O: Water

HCl: Hydrochloric acid

### HPLC: High Performance Liquid Chromatography

KHMDS: Potassium bis(trimethylsilyl)amide
LiHMDS: Lithium bis(trimethylsilyl)amide
NaHMDS: Sodium bis(trimethylsilyl)amide
NaOH: Sodium hydroxide

### N₂: Nitrogen

TBME: t-Butyl methyl ether

### THF: Tetrahydrofuran

Nomenclature for the Example compounds was provided using ACD Name version 5.07 software (Nov. 14, 2001) available from Advanced Chemistry Development, Inc., ChemInnovation NamExpert+ Nomenclator™ brand software available from ChemInnovation Software, Inc., and AutoNom version 2.2 available in the ChemOffice® Ultra software package version 7.0 available from CambridgeSoft Corporation (Cambridge, Mass.). Some of the compounds and starting materials were named using standard IUPAC nomenclature.

Various starting materials may be obtained from commercial sources and prepared by methods known to one of skill in the art.

### Example 1

### Synthesis of 5-(4-Methyl-piperazin-1-yl)-2-nitroaniline

### Procedure A

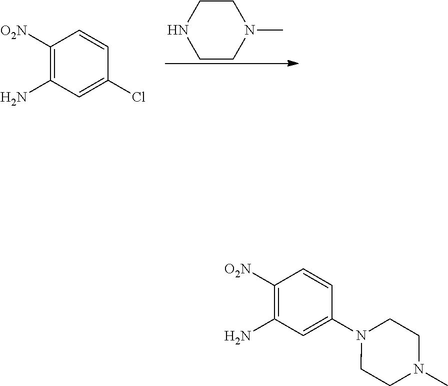

5-Chloro-2-nitroaniline (500 g, 2.898 mol) and 1-methyl piperazine (871 g, 8.693 mol) were placed in a 2000 mL flask fitted with a condenser and purged with N₂. The flask was placed in an oil bath at 100° C. and heated until the 5-chloro-2-nitroaniline was completely reacted (typically overnight) as determined by HPLC. After HPLC confirmed the disappearance of the 5-chloro-2-nitroaniline, the reaction mixture was poured directly (still warm) into 2500 mL of room temperature water with mechanical stirring. The resulting mixture was stirred until it reached room temperature and then it was filtered. The yellow solid thus obtained was added to 1000 mL of water and stirred for 30 minutes. The resulting mixture was filtered, and the resulting solid was washed with TBME (500 mL, 2×) and then was dried under vacuum for one hour using a rubber dam. The resulting solid was transferred to a drying tray and dried in a vacuum oven at 50° C. to a constant weight to yield 670 g (97.8%) of the title compound as a yellow powder.

### Procedure B

5-Chloro-2-nitroaniline (308.2 g, 1.79 mol) was added to a 4-neck 5000 mL round bottom flask fitted with an overhead stirrer, condenser, gas inlet, addition funnel, and thermometer probe. The flask was then purged with N₂. 1-Methylpiperazine (758.1 g, 840 mL, 7.57 mol) and 200 proof ethanol (508 mL) were added to the reaction flask with stirring. The flask was again purged with N₂, and the reaction was maintained under N₂. The flask was heated in a heating mantle to an internal temperature of 97° C. (+/−5° C.) and maintained at that temperature until the reaction was complete (typically about 40 hours) as determined by HPLC. After the reaction was complete, heating was discontinued and the reaction was cooled to an internal temperature of about 20° C. to 25° C. with stirring, and the reaction was stirred for 2 to 3 hours. Seed crystals (0.20 g, 0.85 mmol) of 5-(4-methyl-piperazin-1-yl)-2-nitroaniline were added to the reaction mixture unless precipitation had already occurred. Water (2,450 mL) was added to the stirred reaction mixture over a period of about one hour while the internal temperature was maintained at a temperature ranging from about 20° C. to 30° C. After the addition of water was complete, the resulting mixture was stirred for about one hour at a temperature of 20° C. to 30° C. The resulting mixture was then filtered, and the flask and filter cake were washed with water (3×2.56 L). The golden yellow solid product was dried to a constant weight of 416 g (98.6% yield) under vacuum at about 50° C. in a vacuum oven.

### Procedure C

5-Chloro-2-nitroaniline (401 g, 2.32 mol) was added to a 4-neck 12 L round bottom flask fitted with an overhead stirrer, condenser, gas inlet, addition funnel, and thermometer probe. The flask was then purged with N₂. 1-Methylpiperazine (977 g, 1.08 L, 9.75 mol) and 100% ethanol (650 mL) were added to the reaction flask with stirring. The flask was again purged with N₂, and the reaction was maintained under N₂. The flask was heated in a heating mantle to an internal temperature of 97° C. (+/−5° C.) and maintained at that temperature until the reaction was complete (typically about 40 hours) as determined by HPLC. After the reaction was complete, heating was discontinued and the reaction was cooled to an internal temperature of about 80° C. with stirring, and water (3.15 L) was added to the mixture via an addition funnel over the period of 1 hour while the internal temperature was maintained at 82° C. (+/−3° C.). After water addition was complete, heating was discontinued and the reaction mixture was allowed to cool over a period of no less than 4 hours to an internal temperature of 20-25° C. The reaction mixture was then stirred for an additional hour at an internal temperature of 20-30° C. The resulting mixture was then filtered, and the flask and filter cake were washed with water (1×1 L), 50% ethanol (1×1 L), and 95% ethanol (1×1 L). The golden yellow solid product was placed in a drying pan and dried to a constant weight of 546 g (99% yield) under vacuum at about 50° C. in a vacuum oven.

### Example 2

### Synthesis of [6-(4-Methyl-piperazin-1-yl)-1H-benzimidazol-2-yl]-acetic acid ethyl ester

### Procedure A

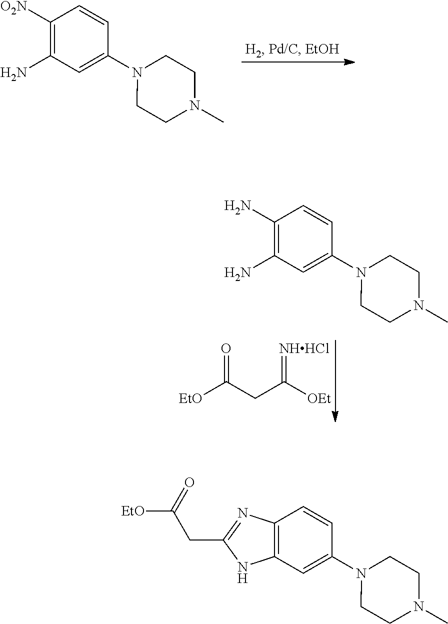

A 5000 mL, 4-neck flask was fitted with a stirrer, thermometer, condenser, and gas inlet/outlet. The equipped flask was charged with 265.7 g (1.12 mol. 1.0 eq) of 5-(4-methyl-piperazin-1-yl)-2-nitroaniline and 2125 mL of 200 proof EtOH. The resulting solution was purged with N₂ for 15 minutes. Next, 20.0 g of 5% Pd/C (50% H₂O w/w) was added. The reaction was vigorously stirred at 40-50° C. (internal temperature) while H₂ was bubbled through the mixture. The reaction was monitored hourly for the disappearance of 5-(4-methyl-piperazin-1-yl)-2-nitroaniline by HPLC. The typical reaction time was 6 hours.

After all the 5-(4-methyl-piperazin-1-yl)-2-nitroaniline had disappeared from the reaction, the solution was purged with N₂ for 15 minutes. Next, 440.0 g (2.25 mol) of ethyl 3-ethoxy-3-iminopropanoate hydrochloride was added as a solid. The reaction was stirred at 40-50° C. (internal temperature) until the reaction was complete. The reaction was monitored by following the disappearance of the diamino compound by HPLC. The typical reaction time was 1-2 hours. After the reaction was complete, it was cooled to room temperature and filtered through a pad of Celite filtering material. The Celite filtering material was washed with absolute EtOH (2×250 mL), and the filtrate was concentrated under reduced pressure providing a thick brown/orange oil. The resulting oil was taken up in 850 mL of a 0.37% HCl solution. Solid NaOH (25 g) was then added in one portion, and a precipitate formed. The resulting mixture was stirred for 1 hour and then filtered. The solid was washed with H₂O (2×400 mL) and dried at 50° C. in a vacuum oven providing 251.7 g (74.1%), of [6-(4-methyl-piperazin-1-yl)-1H-benzoimidazol-2-yl]-acetic acid ethyl ester as a pale yellow powder.

### Procedure B

A 5000 mL, 4-neck jacketed flask was fitted with a mechanical stirrer, condenser, temperature probe, gas inlet, and oil bubbler. The equipped flask was charged with 300 g (1.27 mol) of 5-(4-methyl-piperazin-1-yl)-2-nitroaniline and 2400 mL of 200 proof EtOH (the reaction may be and has been conducted with 95% ethanol and it is not necessary to use 200 proof ethanol for this reaction). The resulting solution was stirred and purged with N₂ for 15 minutes. Next, 22.7 g of 5% Pd/C (50% H₂O w/w) was added to the reaction flask. The reaction vessel was purged with N₂ for 15 minutes. After purging with N₂, the reaction vessel was purged with H₂ by maintaining a slow, but constant flow of H₂ through the flask. The reaction was stirred at 45-55° C. (internal temperature) while H₂ was bubbled through the mixture until the 5-(4-methyl-piperazin-1-yl)-2-nitroaniline was completely consumed as determined by HPLC. The typical reaction time was 6 hours.

After all the 5-(4-methyl-piperazin-1-yl)-2-nitroaniline had disappeared from the reaction, the solution was purged with N₂ for 15 minutes. The diamine intermediate is air sensitive so care was taken to avoid exposure to air. 500 g (2.56 mol) of ethyl 3-ethoxy-3-iminopropanoate hydrochloride was added to the reaction mixture over a period of about 30 minutes. The reaction was stirred at 45-55° C. (internal temperature) under N₂ until the diamine was completely consumed as determined by HPLC. The typical reaction time was about 2 hours. After the reaction was complete, the reaction was filtered while warm through a pad of Celite. The reaction flask and Celite were then washed with 200 proof EtOH (3×285 mL). The filtrates were combined in a 5000 mL flask, and about 3300 mL of ethanol was removed under vacuum producing an orange oil. Water (530 mL) and then 1M HCL (350 mL) were added to the resulting oil, and the resulting mixture was stirred. The resulting solution was vigorously stirred while 30% NaOH (200 mL) was added over a period of about 20 minutes maintaining the internal temperature at about 25-30° C. while the pH was brought to between 9 and 10. The resulting suspension was stirred for about 4 hours while maintaining the internal temperature at about 20-25° C. The resulting mixture was filtered, and the filter cake was washed with H₂O (3×300 mL). The collected solid was dried to a constant weight at 50° C. under vacuum in a vacuum oven providing 345.9 g (90.1%) of [6-(4-methyl-piperazin-1-yl)-1H-benzoimidazol-2-yl]-acetic acid ethyl ester as a pale yellow powder. In an alternative work up procedure, the filtrates were combined and the ethanol was removed under vacuum until at least about 90% had been removed. Water at a neutral pH was then added to the resulting oil, and the solution was cooled to about 0° C. An aqueous 20% NaOH solution was then added slowly with rapid stirring to bring the pH up to 9.2 (read with pH meter). The resulting mixture was then filtered and dried as described above. The alternative work up procedure provided the light tan to light yellow product in yields as high as 97%.

### Example 3

### Method for Reducing Water Content of [6-(4-Methyl-piperazin-1-yl)-1H-benzoimidazol-2-yl]-acetic acid ethyl ester

[6-(4-Methyl-piperazin-1-yl)-1H-benzimidazol-2-yl]-acetic acid ethyl ester (120.7 grams) that had been previously worked up and dried to a water content of about 8-9% H₂O was placed in a 2000 mL round bottom flask and dissolved in absolute ethanol (500 mL). The amber solution was concentrated to a thick oil using a rotary evaporator with heating until all solvent was removed. The procedure was repeated two more times. The thick oil thus obtained was left in the flask and placed in a vacuum oven heated at 50° C. overnight. Karl Fisher analysis results indicated a water content of 5.25%. The lowered water content obtained by this method provided increased yields in the procedure of Example 4. Other solvents such as toluene and THF may be used in place of the ethanol for this drying process.

### Example 4

### Synthesis of 4-Amino-5-fluoro-3-[6-(4-methyl-piperazin-1-yl)-1H-benzimidazol-2-yl]-1H-quinolin-2-one

### Procedure A

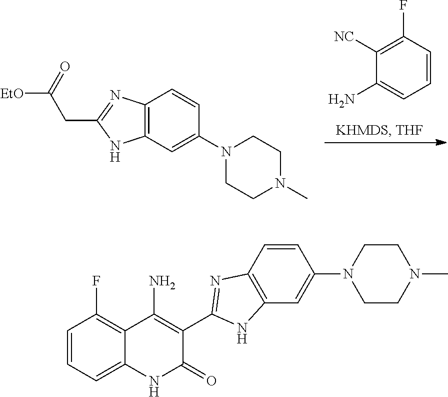

[6-(4-Methyl-piperazin-1-yl)-1H-benzimidazol-2-yl]-acetic acid ethyl ester (250 g, 820 mmol) (dried with ethanol as described above) was dissolved in THF (3800 mL) in a 5000 mL flask fitted with a condenser, mechanical stirrer, temperature probe, and purged with argon. 2-Amino-6-fluoro-benzonitrile (95.3 g, 700 mmol) was added to the solution, and the internal temperature was raised to 40° C. When all the solids had dissolved and the solution temperature had reached 40° C., solid KHMDS (376.2 g, 1890 mmol) was added over a period of 5 minutes. When addition of the potassium base was complete, a heterogeneous yellow solution was obtained, and the internal temperature had risen to 62° C. After a period of 60 minutes, the internal temperature decreased back to 40° C., and the reaction was determined to be complete by HPLC (no starting material or uncyclized intermediate was present). The thick reaction mixture was then quenched by pouring it into H₂O (6000 mL) and stirring the resulting mixture until it had reached room temperature. The mixture was then filtered, and the filter pad was washed with water (1000 mL 2×). The bright yellow solid was placed in a drying tray and dried in a vacuum oven at 50° C. overnight providing 155.3 g (47.9%) of the desired 4-amino-5-fluoro-3-[6-(4-methyl-piperazin-1-yl)-1H-benzimidazol-2-yl]-1H-quinolin-2-one.

### Procedure B

A 5000 mL 4-neck jacketed flask was equipped with a distillation apparatus, a temperature probe, a N₂ gas inlet, an addition funnel, and a mechanical stirrer. [6-(4-Methyl-piperazin-1-yl)-1H-benzimidazol-2-yl]-acetic acid ethyl ester (173.0 g, 570 mmol) was charged into the reactor, and the reactor was purged with N₂ for 15 minutes. Dry THF (2600 mL) was then charged into the flask with stirring. After all the solid had dissolved, solvent was removed by distillation (vacuum or atmospheric (the higher temperature helps to remove the water) using heat as necessary. After 1000 mL of solvent had been removed, distillation was stopped and the reaction was purged with N₂. 1000 mL of dry THF was then added to the reaction vessel, and when all solid was dissolved, distillation (vacuum or atmospheric) was again conducted until another 1000 mL of solvent had been removed. This process of adding dry THF and solvent removal was repeated at least 4 times (on the 4^{th} distillation, 60% of the solvent is removed instead of just 40% as in the first 3 distillations) after which a 1 mL sample was removed for Karl Fischer analysis to determine water content. If the analysis showed that the sample contained less than 0.20% water, then reaction was continued as described in the next paragraph. However, if the analysis showed more than 0.20% water, then the drying process described above was continued until a water content of less than 0.20% was achieved.

After a water content of less than or about 0.20% was achieved using the procedure described in the previous paragraph, the distillation apparatus was replaced with a reflux condenser, and the reaction was charged with 2-amino-6-fluoro-benzonitrile (66.2 g, 470 mmol) (in some procedures 0.95 equivalents is used). The reaction was then heated to an internal temperature of 38-42° C. When the internal temperature had reached 38-42° C., KHMDS solution (1313 g, 1.32 mol, 20% KHMDS in THF) was added to the reaction via the addition funnel over a period of 5 minutes maintaining the internal temperature at about 38-50° C. during the addition. When addition of the potassium base was complete, the reaction was stirred for 3.5 to 4.5 hours (in some examples it was stirred for 30 to 60 minutes and the reaction may be complete within that time) while maintaining the internal temperature at from 38-42° C. A sample of the reaction was then removed and analyzed by HPLC. If the reaction was not complete, additional KHMDS solution was added to the flask over a period of 5 minutes and the reaction was stirred at 38-42° C. for 45-60 minutes (the amount of KHMDS solution added was determined by the following: If the IPC ratio is <3.50, then 125 mL was added; if 10.0≧IPC ratio≧3.50, then 56 mL was added; if 20.0≧IPC ratio≧10, then 30 mL was added. The IPC ratio is equal to the area corresponding to 4-amino-5-fluoro-3-[6-(4-methyl-piperazin-1-yl)-1H-benzimidazol-2-yl]-1H-quinolin-2-one) divided by the area corresponding to the uncyclized intermediate). Once the reaction was complete (IPC ratio>20), the reactor was cooled to an internal temperature of 25-30° C., and water (350 mL) was charged into the reactor over a period of 15 minutes while maintaining the internal temperature at 25-35° C. (in one alternative, the reaction is conducted at 40° C. and water is added within 5 minutes. The quicker quench reduces the amount of impurity that forms over time). The reflux condenser was then replaced with a distillation apparatus and solvent was removed by distillation (vacuum or atmospheric) using heat as required. After 1500 mL of solvent had been removed, distillation was discontinued and the reaction was purged with N₂. Water (1660 mL) was then added to the reaction flask while maintaining the internal temperature at 20-30° C. The reaction mixture was then stirred at 20-30° C. for 30 minutes before cooling it to an internal temperature of 5-10° C. and then stirring for 1 hour. The resulting suspension was filtered, and the flask and filter cake were washed with water (3×650 mL). The solid thus obtained was dried to a constant weight under vacuum at 50° C. in a vacuum oven to provide 103.9 g (42.6% yield) of 4-amino-5-fluoro-3-[6-(4-methyl-piperazin-1-yl)-1H-benzimidazol-2-yl]-1H-quinolin-2-one as a yellow powder.

### Procedure C

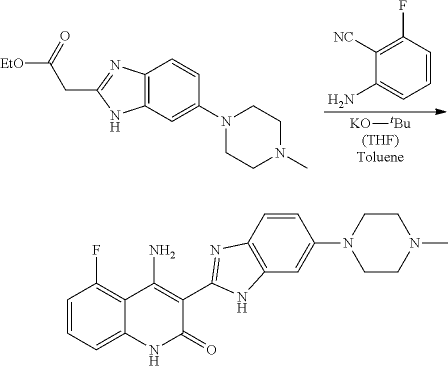

[6-(4-Methyl-piperazin-1-yl)-1H-benzimidazol-2-yl]-acetic acid ethyl ester (608 g, 2.01 mol) (dried) and 2-amino-6-fluoro-benzonitrile (274 g, 2.01 mol) were charged into a 4-neck 12 L flask seated on a heating mantle and fitted with a condenser, mechanical stirrer, gas inlet, and temperature probe. The reaction vessel was purged with N₂, and toluene (7.7 L) was charged into the reaction mixture while it was stirred. The reaction vessel was again purged with N₂ and maintained under N₂. The internal temperature of the mixture was raised until a temperature of 63° C. (+/−3° C.) was achieved. The internal temperature of the mixture was maintained at 63° C. (+/−3° C.) while approximately 2.6 L of toluene was distilled from the flask under reduced pressure (380+/−10 torr, distilling head t=40° C. (+/−10° C.) (Karl Fischer analysis was used to check the water content in the mixture. If the water content was greater than 0.03%, then another 2.6 L of toluene was added and distillation was repeated. This process was repeated until a water content of less than 0.03% was achieved). After a water content of less than 0.03% was reached, heating was discontinued, and the reaction was cooled under N₂ to an internal temperature of 17-19° C. Potassium t-butoxide in THF (20% in THF; 3.39 kg, 6.04 moles potassium t-butoxide) was then added to the reaction under N₂ at a rate such that the internal temperature of the reaction was kept below 20° C. After addition of the potassium t-butoxide was complete, the reaction was stirred at an internal temperature of less than 20° C. for 30 minutes. The temperature was then raised to 25° C., and the reaction was stirred for at least 1 hour. The temperature was then raised to 30° C., and the reaction was stirred for at least 30 minutes. The reaction was then monitored for completion using HPLC to check for consumption of the starting materials (typically in 2-3 hours, both starting materials were consumed (less than 0.5% by area % HPLC)). If the reaction was not complete after 2 hours, another 0.05 equivalents of potassium t-butoxide was added at a time, and the process was completed until HPLC showed that the reaction was complete. After the reaction was complete, 650 mL of water was added to the stirred reaction mixture. The reaction was then warmed to an internal temperature of 50° C. and the THF was distilled away (about 3 L by volume) under reduced pressure from the reaction mixture. Water (2.6 L) was then added dropwise to the reaction mixture using an addition funnel. The mixture was then cooled to room temperature and stirred for at least 1 hour. The mixture was then filtered, and the filter cake was washed with water (1.2 L), with 70% ethanol (1.2 L), and with 95% ethanol (1.2 L). The bright yellow solid was placed in a drying tray and dried in a vacuum oven at 50° C. until a constant weight was obtained providing 674 g (85.4%) of the desired 4-amino-5-fluoro-3-[6-(4-methyl-piperazin-1-yl)-1H-benzimidazol-2-yl]-1H-quinolin-2-one.

### Example 5

### Purification of 4-Amino-5-fluoro-3-[6-(4-methyl-piperazin-1-yl)-1H-benzimidazol-2-yl]-1H-quinolin-2-one

A 3000 mL 4-neck flask equipped with a condenser, temperature probe, N₂ gas inlet, and mechanical stirrer was placed in a heating mantle. The flask was then charged with 4-amino-5-fluoro-3-[6-(4-methyl-piperazin-1-yl)-1H-benzimidazol-2-yl]-1H-quinolin-2-one (101.0 g, 0.26 mol), and the yellow solid was suspended in 95% ethanol (1000 mL) and stirred. In some cases an 8:1 solvent ratio is used. The suspension was then heated to a gentle reflux (temperature of about 76° C.) with stirring over a period of about 1 hour. The reaction was then stirred for 45-75 minutes while refluxed. At this point, the heat was removed from the flask and the suspension was allowed to cool to a temperature of 25-30° C. The suspension was then filtered, and the filter pad was washed with water (2×500 mL). The yellow solid was then placed in a drying tray and dried in a vacuum oven at 50° C. until a constant weight was obtained (typically 16 hours) to obtain 97.2 g (96.2%) of the purified product as a yellow powder.

### Example 6

### Preparation of Lactic Acid salt of 4-Amino-5-fluoro-3-[6-(4-methyl-piperazin-1-yl)-1H-benzimidazol-2-yl]-1H-quinolin-2-one

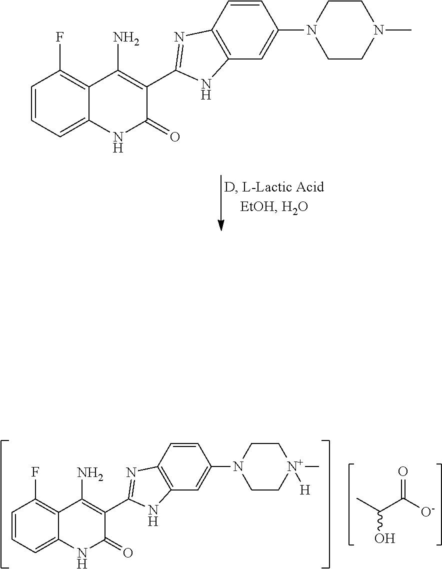

A 3000 mL 4-necked jacketed flask was fitted with a condenser, a temperature probe, a N₂ gas inlet, and a mechanical stirrer. The reaction vessel was purged with N₂ for at least 15 minutes and then charged with 4-amino-5-fluoro-3-[6-(4-methyl-piperazin-1-yl)-1H-benzimidazol-2-yl]-1H-quinolin-2-one (484 g, 1.23 mol). A solution of D,L-Lactic acid (243.3 g, 1.72 mol of monomer-see the following paragraph), water (339 mL), and ethanol (1211 mL) was prepared and then charged to the reaction flask. Stirring was initiated at a medium rate, and the reaction was heated to an internal temperature of 68-72° C. The internal temperature of the reaction was maintained at 68-72° C. for 15-45 minutes and then heating was discontinued. The resulting mixture was filtered through a 10-20 micron frit collecting the filtrate in a 12 L flask. The 12 L flask was equipped with an internal temperature probe, a reflux condenser, an addition funnel, a gas inlet an outlet, and an overhead stirrer. The filtrate was then stirred at a medium rate and heated to reflux (internal temperature of about 78° C.). While maintaining a gentle reflux, ethanol (3,596 mL) was charged to the flask over a period of about 20 minutes. The reaction flask was then cooled to an internal temperature ranging from about 64-70° C. within 15-25 minutes and this temperature was maintained for a period of about 30 minutes. The reactor was inspected for crystals. If no crystals were present, then crystals of the lactic acid salt of 4-amino-5-fluoro-3-[6-(4-methyl-piperazin-1-yl)-1H-benzimidazol-2-yl]-1H-quinolin-2-one (484 mg, 0.1 mole %) were added to the flask, and the reaction was stirred at 64-70° C. for 30 minutes before again inspecting the flask for crystals. Once crystals were present, stirring was reduced to a low rate and the reaction was stirred at 64-70° C. for an additional 90 minutes. The reaction was then cooled to about 0° C. over a period of about 2 hours, and the resulting mixture was filtered through a 25-50 micron fritted filter. The reactor was washed with ethanol (484 mL) and stirred until the internal temperature was about 0° C. The cold ethanol was used to wash the filter cake, and this procedure was repeated 2 more times. The collected solid was dried to a constant weight at 50° C. under vacuum in a vacuum oven yielding 510.7 g (85.7%) of the crystalline yellow lactic acid salt of 4-amino-5-fluoro-3-[6-(4-methyl-piperazin-1-yl)-1H-benzimidazol-2-yl]-1H-quinolin-2-one. A rubber dam or inert conditions were typically used during the filtration process. While the dry solid did not appear to be very hygroscopic, the wet filter cake tends to pick up water and become sticky. Precautions were taken to avoid prolonged exposure of the wet filter cake to the atmosphere.

Commercial lactic acid generally contains about 8-12% w/w water, and contains dimers and trimers in addition to the monomeric lactic acid. The mole ratio of lactic acid dimer to monomer is generally about 1.0:4.7. Commercial grade lactic acid may be used in the process described in the preceding paragraph as the monolactate salt preferentially precipitates from the reaction mixture.

It should be understood that the organic compounds according to the invention may exhibit the phenomenon of tautomerism. As the chemical structures within this specification can only represent one of the possible tautomeric forms at a time, it should be understood that the invention encompasses any tautomeric form of the drawn structure. For example, the compound having the formula IIIB is shown below with one tautomer, Tautomer IIIBa:

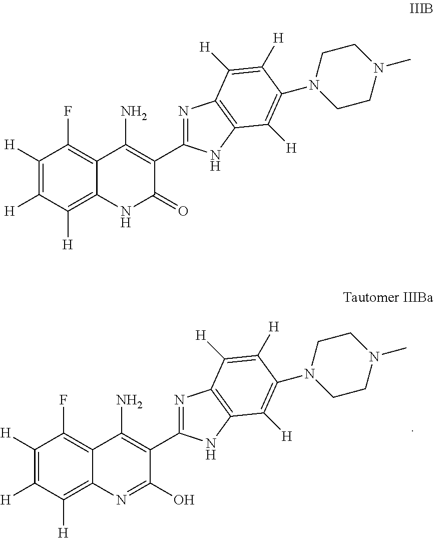

Other tautomers of the compound having the formula IIIB, Tautomer IIIIBb and Tautomer IIIIBc, are shown below:

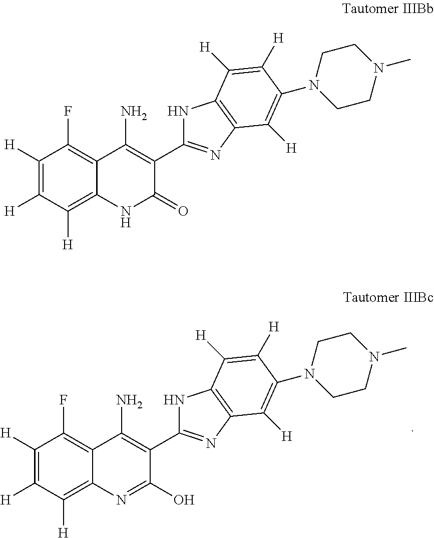

The contents of each of the patents, patent applications and journal articles cited above are hereby incorporated by reference herein and for all purposes as if fully set forth in their entireties.

It is understood that the invention is not limited to the embodiments set forth herein for illustration, but embraces all such forms thereof as come within the scope of the following claims.

What is claimed is:

1. A method of synthesizing a substituted or unsubstituted benzimidazolyl quinolinone compound, comprising: reacting a first compound having the formula I with a second compound having the formula II in a suitable solvent in the presence of a sodium or potassium salt of a base to provide a reaction product comprising the benzimidazolyl quinolinone compound, wherein the first compound and the second compound have the following structures 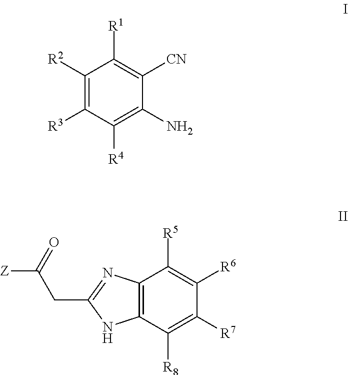
wherein
R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;
R⁵, R⁶, R⁷, and R⁸ may be the same or different and are independently selected from H, Cl, Br, F, 1, —OR¹³ groups, —NR¹⁴R¹⁵ groups, —SR¹⁶ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
Z is selected from —OR^{9a} groups or —NR^{9b}R^{9c} groups;
R^{9a} is an unsubstituted alkyl group having from 1 to 8 carbon atoms and is absent if Z is a —NR^{9b}R^{9c} group;
R^{9b} and R^{9c} are independently selected from unsubstituted alkyl groups having from 1 to 8 carbon atoms or are both absent if Z is a —OR^{9a} group;
R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
R¹¹ and R¹⁴ may be the same or different and are independently selected from substituted or unsubstituted alkyl groups, substituted or unsubstituted aryl groups, or substituted or unsubstituted heterocyclyl groups;
R¹² and R¹⁵ may be the same or different and are independently selected from substituted or unsubstituted alkyl groups, substituted or unsubstituted aryl groups, or substituted or unsubstituted heterocyclyl groups; and
R¹⁶ is selected from substituted or unsubstituted alkyl groups, substituted or unsubstituted aryl groups, or substituted or unsubstituted heterocyclyl groups; and
further wherein the substituted or unsubstituted benzimidazolyl compound is a compound having the formula III, is a tautomer of the compound having the formula III, is a salt of the compound having the formula III, or is a salt of the tautomer of the compound having the formula III

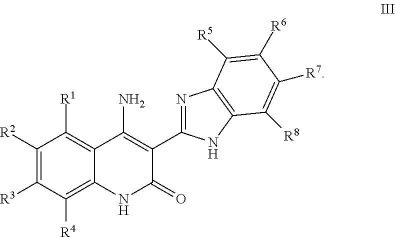

2. The method of claim 1, wherein R¹ is selected from H, Cl, Br, F, or I.

3. The method of claim 1, wherein R¹ is F.

4. The method of claim 1, wherein R², R³, and R⁴ are all H.

5. The method of claim 1, wherein the first compound is a compound having the formula IA having the following structure

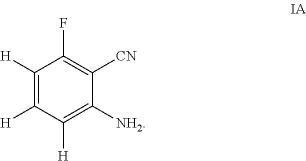

6. The method of claim 1, wherein at least one of R⁶ or R⁷ is a substituted or unsubstituted heterocyclyl group.

7. The method of claim 1, wherein one of R⁶ or R⁷ is a substituted or unsubstituted heterocyclyl group and the other of R⁶ or R⁷ is a H.

8. The method of claim 1, wherein one of R⁶ or R⁷ is a substituted or unsubstituted heterocyclyl group selected from a substituted or unsubstituted piperidinyl group, piperazinyl group, or morpholinyl group.

9. The method of claim 8, wherein one of R⁶ or R⁷ is a substituted or unsubstituted piperazinyl group.

10. The method of claim 9, wherein one of R⁶ or R⁷ is an N-alkyl piperazinyl group.

11. The method of claim 10, wherein one of R⁶ or R⁷ is an N-methyl piperazinyl group and the other of R⁶ or R⁷ is an H.

12. The method of claim 1, wherein the second compound is a compound having the formula IIA or IIB 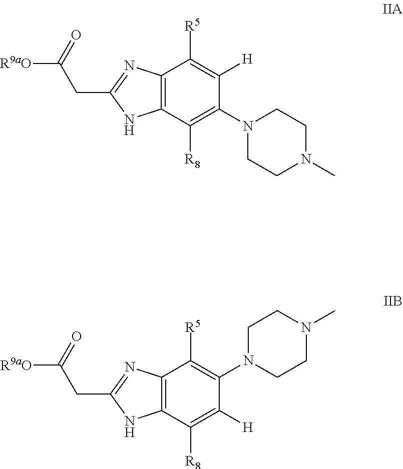
and R⁵, R⁸, and R^{9a} have the values defined in claim 1.

13. The method of claim 1, wherein the second compound is a compound having the formula IIC or IID 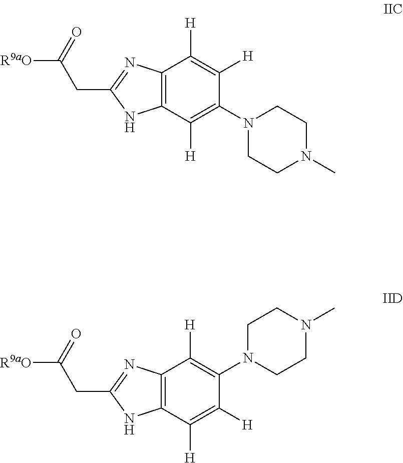
where R^{9a} has the values defined in claim 1.

14. The method of claim 13, wherein R^{9a} is a straight or branched chain alkyl group selected from methyl, ethyl, propyl, butyl, pentyl, i-propyl, s-butyl, or t-butyl groups.

15. The method of claim 14, wherein R^{9a} is an ethyl group.

16. The method of claim 1, wherein the suitable solvent is selected from a dialkyl ether, a cyclic ether, an aromatic solvent, or a combination thereof.

17. The method of claim 16, wherein the solvent is tetrahydrofuran.

18. The method of claim 1, wherein the solvent is toluene.

19. The method of claim 1, wherein the sodium or potassium salt of the base is a sodium or potassium alkoxide.

20. The method of claim 19, wherein the sodium or potassium salt of the base is potassium t-butoxide.

21. The method of claim 1, wherein the sodium or potassium salt of the base is a sodium or potassium bis(trialkylsilyl)amide.

22. The method of claim 21, wherein the sodium or potassium bis(trialkylsilyl)amide is sodium bis(trimethylsilyl)amide or potassium bis(trimethylsilyl)amide.

23. The method of claim 16, further comprising adding the sodium or potassium salt of the base to a mixture of the first compound and the second compound in the suitable solvent.

24. The method claim 1, wherein the sodium or potassium salt of the base is present in an amount of from 2 to 4 equivalents with respect to the molar quantity of the first compound.

25. The method of claim 1, wherein the sodium or potassium salt of the base is present in an amount of from 2 to 4 equivalents with respect to the molar quantity of the second compound.

26. The method of claim 1, wherein the second compound is present in an amount of from 1 to 2 equivalents with respect to the molar quantity of the first compound.

27. The method of claim 1, further comprising adding the sodium or potassium salt of the base to a mixture comprising the first compound, the second compound, and the suitable solvent at a temperature of from 15° C. to 50° C.

28. The method of claim 1, further comprising (a) adding an aromatic solvent to a reaction flask to provide a reaction mixture comprising the solvent, the first compound, and the second compound, (b) distilling a portion of the aromatic solvent from the reaction flask, and (c) repeating (a) and (b) until the water content of the reaction mixture is less than 0.05%.

29. The method of claim 1, wherein the second compound is placed in a reaction flask and dried by (a) adding THF to the reaction flask to create a reaction mixture, (b) distilling a portion of the THF from the reaction flask, and (c) repeating (a) and (b) until the water content of the reaction mixture is less than 0.5%.

30. The method of claim 29, further comprising repeating (a) and (b) until the water content of the reaction mixture is less than or equal to 0.2%.

31. The method of claim 1, wherein the second compound is dried by: (a) mixing the second compound with an organic solvent to form a solution; (b) removing a portion of the organic solvent to provide the dried second compound; (c) optionally repeating (a) and (b) one or more additional times; and (d) additionally drying the dried second compound by heating it under a vacuum.

32. The method of claim 1, wherein the first compound is reacted with the second compound in the presence of the sodium or potassium salt of the base for a period of from 30 to 360 minutes.

33. The method of claim 1, further comprising mixing the reaction product comprising the benzimidazolyl quinolinone compound with water to provide a quenched reaction mixture.

34. The method of claim 33, further comprising filtering the quenched reaction mixture and washing it with water to provide a collected product.

35. The method of claim 34, further comprising (a) mixing the collected product with ethanol to provide an ethanolic mixture of the collected product; (b) heating the ethanolic mixture of the collected product for a period of from 10 minutes to 180 minutes at a temperature of from 40° C. to 78° C.; (c) cooling the ethanolic mixture of the collected product to a temperature of less than 40° C.; (d) and filtering the cooled ethanolic mixture of the collected product.

36. The method of claim 1, wherein the benzimidazolyl quinolinone compound is prepared in a yield greater than 40 percent.

37. The method of claim 1, further comprising reacting a compound having the formula IV with a compound having the formula V to prepare the second compound, wherein the compound having the formula IV and the compound having the formula V have the following structures, 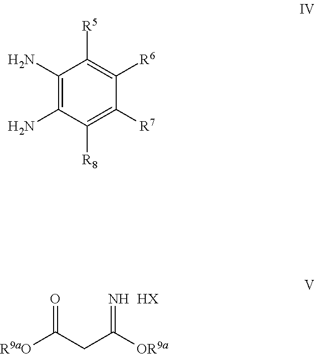
wherein the variables R⁵, R⁶, R⁷, R⁸, and R^{9a} have the values defined in claim 1 and X is a halogen atom selected from F, Cl, Br, or I, or is the conjugate base of an acid.

38. The method of claim 37, wherein the compound having the formula IV is a compound having the formula IVA

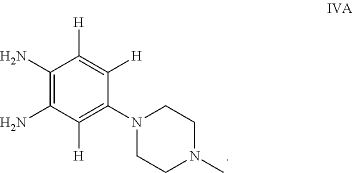

39. The method of claim 37, wherein the compound having the formula V is a compound having the formula VA

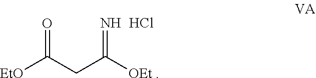

40. The method of claim 37, wherein the compound having the formula IV is reacted with the compound having the formula V in an alcohol solvent at an internal temperature of from 30° C. to 70° C. for a period of time of from 45 minutes to 240 minutes to prepare the second compound.

41. The method of claim 37, further comprising reducing a compound having the formula VIA, VIB, or a mixture thereof to produce the compound having the formula IV 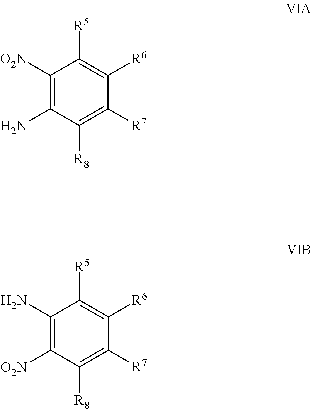
and the variables R⁵, R⁶, R⁷, and R⁸ have the values defined in claim 1.

42. The method of claim 41, wherein H₂ and a hydrogenation catalyst are used to reduce the compound having the formula VIA, the compound having the formula VIB, or the mixture thereof.

43. The method of claim 42, wherein the hydrogenation catalyst comprises palladium on carbon.

44. The method of claim 41, wherein the compound having the formula VIA is a compound having the formula VIC or VID and/or the compound having the formula VIB is a compound having the formula VIE or VIF

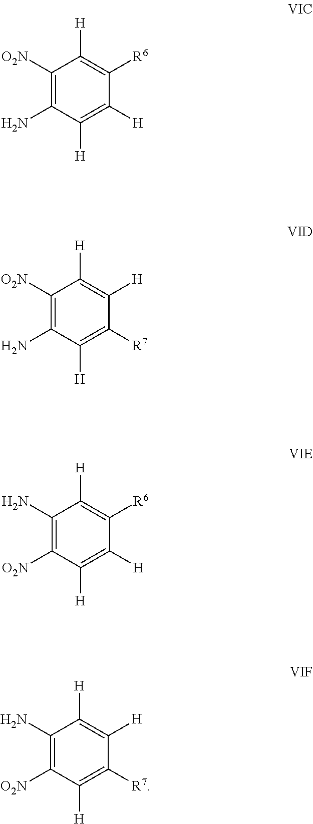

45. The method of claim 44, wherein R⁶ and R⁷ are selected from substituted or unsubstituted heterocyclyl groups.

46. The method of claim 45, wherein R⁶ and R⁷ are selected from substituted or unsubstituted piperidinyl groups, piperazinyl groups, or morpholinyl groups.

47. The method of claim 46, wherein one of R⁶ or R⁷ is an N-alkyl piperazinyl group.

48. The method of claim 47, wherein one of R⁶ or R⁷ is an N-methyl piperazinyl group such that the compound having the formula VIC, VID, VIE, or VIF is a compound having the formula VIG or VIH

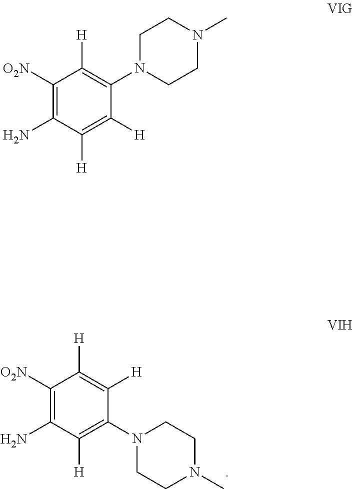

49. The method of claim 48, wherein the compound reduced to provide the compound having the formula IV is the compound having the formula VIH.

50. The method of claim 41, further comprising reacting a compound having the formula VII with a compound having the formula HR⁷ or a salt thereof to prepare the compound having the formula VIA, 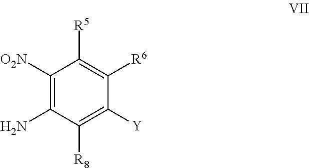
wherein the variables R⁵, R⁶, R⁷, and R⁸ have the values defined in claim 1 and Y is selected from Cl or F.

51. The method of claim 50, wherein the compound having the formula VII is a compound having the formula VIIA or VIIB

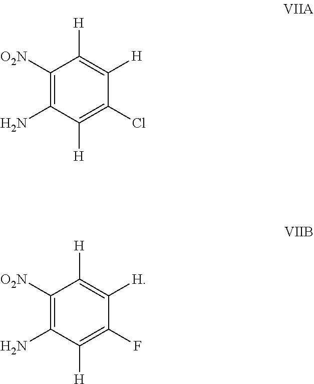

52. The method of claim 50, wherein R⁷ is a substituted or unsubstituted heterocyclyl group.

53. The method of claim 52, wherein R⁷ is an N-alkyl piperazinyl group.

54. The method of claim 52, wherein R⁷ is an N-methyl piperazinyl group and HR⁷ is a compound having the formula HR⁷(a)

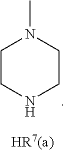

55. The method of claim 52, wherein the compound having the formula VII and the compound having the formula HR⁷ are reacted at a temperature of from 70° C. to 120° C. for a period of from 2 hours to 24 hours to prepare the compound having the formula VIA.

56. The method of claim 1, wherein the benzimidazolyl quinolinone compound is a compound having the formula IIIA, is a tautomer of the compound having the formula IIIA, is a salt of the compound having the formula IIIA, or is a salt of the tautomer of the compound having the formula IIIA and R⁷ is a substituted or unsubstituted heterocyclyl group

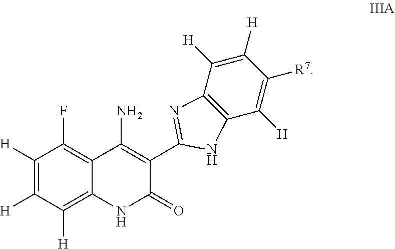

57. The method of claim 56, wherein R⁷ is a substituted or unsubstituted heterocyclyl group selected from a substituted or unsubstituted piperidinyl group, piperazinyl group, or morpholinyl group.

58. The method of claim 57, wherein R⁷ is a substituted or unsubstituted N-alkyl piperazinyl group.

59. The method of claim 1, wherein the benzimidazolyl quinolinone compound is a compound having the formula IIIB, is a tautomer of the compound having the formula IIIB, is a salt of the compound having the formula IIIB, or is a salt of the tautomer of the compound having the formula IIIB

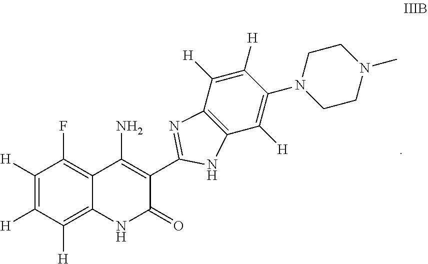

60. The method of claim 59, further comprising reacting the benzimidazolyl quinolinone compound with lactic acid to provide the lactic acid salt of the benzimidazolyl quinolinone compound.

61. The method of claim 60, wherein the lactic acid and the benzimidazolyl quinolinone compound are reacted in a mixture of water and ethanol.

62. A composition comprising a benzimidazolyl quinolinone compound having the formula III, a tautomer of the benzimidazolyl quinolinone compound, a salt of the benzimidazolyl quinolinone compound, a salt of the tautomer of the benzimidazolyl compound, or mixtures thereof, wherein the benzimidazolyl quinolinone compound is a compound having the formula III, 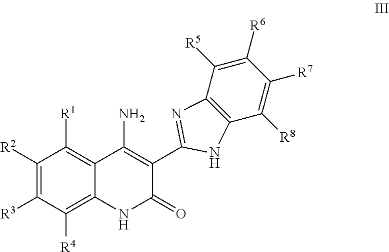
wherein:
R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;
R⁵, R⁶, R⁷, and R⁸ may be the same or different and are independently selected from H, Cl, Br, F, I, —OR¹³ groups, —NR¹⁴R¹⁵ groups, —SR¹⁶ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;
R¹¹ and R¹⁴ may be the same or different and are independently selected from substituted or unsubstituted alkyl groups, substituted or unsubstituted aryl groups, or substituted or unsubstituted heterocyclyl groups;
R¹² and R¹⁵ may be the same or different and are independently selected from substituted or unsubstituted alkyl groups, substituted or unsubstituted aryl groups, or substituted or unsubstituted heterocyclyl groups;
R¹⁶ is selected from substituted or unsubstituted alkyl groups, substituted or unsubstituted aryl groups, or substituted or unsubstituted heterocyclyl groups; and further wherein,
the amount of lithium in the composition is less than 1 percent by weight based on the weight of the benzimidazolyl quinolinone compound in the composition.

63. The composition of claim 62, wherein the amount of lithium in the composition is less than 0.5 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

64. The composition of claim 62, wherein the amount of lithium in the composition is less than 0.1 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

65. The composition of claim 62, wherein the amount of lithium in the composition is less than 0.05 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

66. The composition of claim 62, wherein the amount of lithium in the composition is less than 0.01 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

67. The composition of claim 62, wherein the amount of lithium in the composition is less than 0.005 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

68. The composition of claim 62, wherein the amount of lithium in the composition is less than 0.001 percent by weight based on the weight of the benzimidazolyl quinolinone compound, the tautomer of the benzimidazolyl quinolinone compound, the salt of the benzimidazolyl quinolinone compound, the salt of the tautomer of the benzimidazolyl compound, or the mixtures thereof in the composition.

69. The composition of claim 62, wherein lithium is absent from the composition.

70. The composition of claim 62, wherein the benzimidazolyl quinolinone compound having the formula III is a compound having the formula IIIB

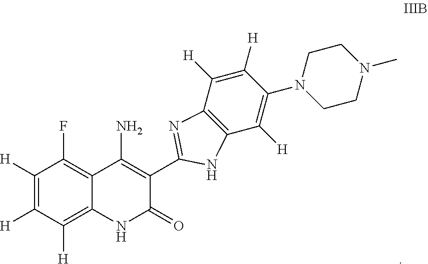

71. The composition of claim 70, wherein the composition comprises the lactic acid salt of the compound having the formula IIIB.
